(19) United States
(12) Patent Application Publication
(10) Pub. No.: US 2026/0157701 A1
(43) Pub. Date: Jun. 11, 2026

(54) SIGNAL PROCESSING UNIT AND PROCESS FOR DETERMINING A RESPIRATORY SIGNAL

(71) Applicant: Drägerwerk AG & Co. KGaA, Lübeck (DE)

(72) Inventor: Lorenz KAHL, Lübeck (DE)

(21) Appl. No.: 19/179,209

(22) Filed: Apr. 15, 2025

(30) Foreign Application Priority Data
Apr. 19, 2024 (DE) 10 2024 111 079.1

(51) Int. Cl. A61B 5/00; A61B 5/08; G16H 50/30

(52) U.S. Cl. CPC A61B 5/7203; A61B 5/0816; A61B 5/7221; A61B 5/7246; A61B 5/7278; A61B 5/7289; G16H 50/30

(57) ABSTRACT

A signal processing unit and process determine an estimate (Sig_{res,est}) for a respiratory signal, which is an indicator of a patient's own spontaneous breathing activity and/or artificial ventilation. A sum signal (Sig_{Sum})—a respiratory and cardiogenic signal, is generated from measured values, measured in and/or on the patient's body. An intermediate signal (Sig_{com}) is calculated by compensating for the influence of cardiac activity on the sum signal. A reference attenuation signal segment describes the average time course of the contribution of the cardiogenic signal to the intermediate signal. An intermediate signal segment [SigA_{com}(x)] is generated for each detected heartbeat. An attenuated intermediate signal segment [SigA_{com,d}(x)] is generated from this, for which an adapted attenuation signal segment [Mod(1)(x), . . . , Mod(n)(x)] is applied to the intermediate signal segment. The adapted attenuation signal segment is calculated from the reference attenuation signal segment using at least one quality indicator [Q(30), Q(31)].

### CROSS REFERENCE TO RELATED APPLICATIONS

This application claims the benefit of priority under 35 U.S.C. § 119 of German Application 10 2024 111 079.1, filed Apr. 19, 2024, the entire contents of which are incorporated herein by reference.

### TECHNICAL FIELD

The invention relates to a signal processing unit and a process which determine an estimate for (indication of/representation of) a respiratory signal. This respiratory signal is an indicator of a patient's own breathing activity (respiratory activity) and/or artificial ventilation (also known as artificial respiration) of the patient. The patient's own breathing activity is caused by his/her spontaneous breathing and/or by external stimulation of his/her respiratory muscles. Both the patient's own breathing activity and artificial ventilation cause ventilation of the patient's lungs. The respiratory signal is required, for example, to determine the condition of the patient's respiratory muscles or to adapt the artificial ventilation to the patient's own breathing activity.

### BACKGROUND

As a rule, the respiratory signal cannot be measured directly. Instead, it is only possible to measure a signal, called a sum signal, that results from a superposition of the respiratory signal sought with a cardiogenic signal and optionally with interfering signals. The cardiogenic signal is an indicator of the patient's cardiac activity.

Prior art signal processing units and processing processes have not been able to effectively process signals, such as sum signal sensor device signals, that comprise and present the superposition of a respiratory signal sought with a cardiogenic signal and potentially also with interfering signals so as to present an estimate for or an indication of or a representation of the respiratory signal that is sought.

### SUMMARY

It is an object of the invention to provide a signal processing unit and a process which are able to obtain a respiratory signal from a sum signal better than known signal processing units and processes, wherein the sum signal has been generated from measured values which have been measured on a patient and comprises a superposition of the respiratory signal and a cardiogenic signal and wherein interference can occur during the measurements. With other words: The respiratory signal is to be generated from the sum signal with a higher reliability.

The problem is solved by a signal processing unit with features according to the invention and by a process with features according to the invention. Advantageous embodiments of the signal processing unit according to the invention are, where appropriate, also advantageous embodiments of the process according to the invention and vice versa.

The following describes exemplary embodiments that comprise a signal processor, a signal processor and sensor system, and a process that process sum signal sensor device signals that present the superposition of the respiratory signal sought with a cardiogenic signal and potentially also with interfering signals. The processing is performed to present an indication or representation or estimate of the respiratory signal sought. Therefore, the embodiments of the invention have the capacity to improve the technical field of signal processors and signal processor and sensor systems and processing processes that process signals such as sum signal sensor device signals that present the superposition of the respiratory signal sought with a cardiogenic signal and potentially also with interfering signals to present an indication or representation or estimate of the respiratory signal sought by a novel combination of signal processing features.

The signal processing unit according to the invention and the process according to the invention are capable of determining an estimate for a respiratory signal (also referred to as a representation of or an indication of a respiratory signal). The respiratory signal to be estimated correlates with the ventilation of a patient's lungs, i.e. with the ventilation and deaeration of the lungs. The ventilation of the lungs is generated by the patient's own breathing activity and/or by artificial ventilation. The patient's own breathing activity is generated by the patient's respiratory muscles, usually by his/her spontaneous breathing and in one embodiment additionally or instead by external stimulation of the respiratory muscles, e.g. by artificial ventilation or in a magnetic field.

A reference heartbeat time period and a use phase are specified (predetermined/given) in a computer-evaluable form (a form that can be analyzed and processed by a computer). The reference heartbeat time period can be used to describe the typical course of a heartbeat—more precisely: a cardiogenic signal over the course of a heartbeat time period. The estimate of the respiratory signal is determined in a use phase.

At least one sum signal sensor is capable of measuring a signal that is generated in and/or on the patient's body, for example with the aid of measuring electrodes or with the aid of a measuring instrument in the patient's body. Optionally, several (a plurality of) sum signal sensors are used. In one embodiment, the or a sum signal sensor comprises several (a plurality of) electrodes that are positioned on the patient's skin. The signal generated at or in the patient's body is generated by the patient's own breathing activity and/or artificial ventilation as well as by the patient's cardiac activity.

According to the invention, the signal processing unit generates at least one sum signal. For doing so, it uses (processes) measured values from the or at least one sum signal sensor. Optionally, the signal processing unit generates a respective sum signal for each sum signal sensor. The or each generated sum signal comprises a superposition of the respiratory signal, which is to be determined approximately (estimated), and a cardiogenic signal. The cardiogenic signal describes the patient's cardiac activity. Optionally, at least one interference signal flows into (interferes, influences) the sum signal.

Using the or at least one sum signal, the signal processing unit detects several (a plurality of) heartbeats that the patient performs in the use phase. Furthermore, the signal processing unit detects a characteristic heartbeat time period for each detected heartbeat. The heartbeat takes place during this heartbeat time period. In this heartbeat time period, the sum signal is essentially determined by the cardiogenic signal; and over the entire heartbeat period or at least in partial periods. Outside a heartbeat time period, the sum signal is essentially determined by the respiratory signal. The same applies to the reference heartbeat time period.

The signal processing unit generates an intermediate signal. To generate the intermediate signal, the signal processing unit compensates at least approximately for the influence of the cardiac activity, i.e. the cardiogenic signal, on the sum signal, for example by subtraction. Preferably, the intermediate signal is the result of this compensation by using subtraction of most of or essential portions of the cardiogenic signal.

The signal processing unit determines at least one reference attenuation signal segment (signal section). In a first alternative, the signal processing unit determines (calculates) the reference attenuation signal segment, in a second alternative the signal processing unit determines the reference attenuation signal segment by read access to a data memory. The or each determined reference attenuation signal segment correlates with the average time course of the contribution of the cardiogenic signal to the intermediate signal, namely with the contribution in the specified reference heartbeat time period. The reference attenuation signal segment refers to the reference heartbeat time period and applies at least approximately to a plurality of heartbeat time periods of the patient.

The respiratory signal to be determined relates to the use phase. The following steps are carried out for each detected heartbeat that falls within the use phase:
As already mentioned, a respective characteristic heartbeat time period for the detected heartbeat is detected. The signal processing unit generates an intermediate signal segment as a section of the intermediate signal. The intermediate signal segment lies in the heartbeat time period of this heartbeat and is, for example, the segment of the intermediate signal that falls within the heartbeat time period. For each detected heartbeat a respective intermediate signal segment is therefore generated.
The signal processing unit generates a respective attenuated (damped) intermediate signal segment for each detected heartbeat.
In order to generate the attenuated intermediate signal segment, the signal processing unit applies the reference attenuation signal segment to the to the intermediate signal segment in the first alternative and applies an adapted attenuation signal segment to the intermediate signal segment in the second alternative.

The attenuated intermediate signal segment for a detected heartbeat has the following property: The influence of the cardiogenic signal on the attenuated intermediate signal segment is less than or at most equal to the influence of the cardiogenic signal on the (non-attenuated) intermediate signal segment, but not greater. Ideally, the cardiogenic signal has no influence at all on the attenuated intermediate signal segment.

The signal processing unit assembles (combines) the attenuated intermediate signal segments to form the required estimate for the respiratory signal. The signal processing unit uses the detected characteristic heartbeat times for this combination. As a result, the attenuated intermediate signal segments are assembled or combined with the correct timing. Optionally, gaps between neighboring attenuated intermediate signal segments are connected using corresponding segments of the intermediate signal.

The signal processing unit determines at least one of four quality indicators (quality indexes). Three of the four quality indicators describe the respective reliability of the following determinations and calculations:
the reliability with which the or each used sum signal sensor (a sum signal sensor assembly comprising one or more sum signal sensors) measures the respective measured values and/or the reliability with which the signal processing unit generates the respective sum signal from these measured values,
for at least one heartbeat, preferably for several heartbeats, in the use phase the respective reliability with which the signal processing unit detected the respective characteristic heartbeat time of the heartbeat,
the reliability with which a reference attenuation signal segment enables to computationally compensate for the contribution of the cardiogenic signal to the intermediate signal in the reference heartbeat time period.

The fourth quality indicator evaluates the shape of the respective intermediate signal segment for a heartbeat.

Preferably, the higher the quality indicator, the better the respective rating. If, instead, the quality indicator is smaller the better the rating, the following description must be modified accordingly.

According to the first alternative, the signal processing unit applies the reference attenuation signal segment to the intermediate signal segment. The signal processing unit has previously determined this reference attenuation signal segment using at least one of the above-mentioned four quality indicators, preferably using a random sample and preferably before the use phase. It is possible that the signal processing unit continuously updates the reference attenuation signal segment during the use phase.

According to the second alternative, the signal processing unit applies an adapted attenuation signal segment to the intermediate signal segment. The adapted attenuation signal segment for a detected heartbeat is generated by the following steps:

The signal processing unit determines the adapted attenuation signal segment, for which it uses the determined, i.e. calculated or read-accessed, reference attenuation signal segment and at least one calculated quality indicator.

The determination is made as follows:
The adapted attenuation signal segment is smaller than or at most the same size as the reference attenuation signal segment.
The smaller is the calculated and applied quality indicator, the smaller is the adapted attenuation signal segment.

The actual cardiogenic signal is referred to as Sigₖₐᵣ, the actual respiratory signal as Sigᵣₑₛ. The estimate for the respiratory signal Sigᵣₑₛ generated according to the invention is Sig_{res,est}. In many cases, an indicator Paw (pressure in airway) for the airway pressure and/or an indicator Pₑₛ (pressure in esophagus) for the esophageal pressure can be derived from measured values provided by optional additional sensors. These indicators can be used to derive a pneumatic indicator Pₘᵤₛ, which is also an indicator of the patient's own breathing activity. By determining an estimate Sig_{res,est} for the electrical or mechanical respiratory signal Sigᵣₑₛ on the one hand and a pneumatic indicator Pₘᵤₛ on the other, the patient's own breathing activity is determined with greater reliability than when only one signal is derived, and it is possible to deduce how well the patient's respiratory muscles convert electrical stimuli generated in the patient's body into pneumatic breathing activity (neuromechanical efficiency). The invention can also be used in an embodiment in which the EMG signal or the MMG signal is generated, but not the pneumatic indicator Pₘᵤₛ for breathing activity.

The estimated respiratory signal Sig_{res,est} determined according to the invention is used, for example, for the following purposes, which are or relate to therapeutic purposes:
The neuromechanical efficiency of the patient's respiratory muscles is determined,
The condition of the patient's respiratory muscles is determined (in particular a fatigue determination)—the pneumatic indicator Pₘᵤₛ is not required for this,
Asynchronies in the patient's own breathing activity are detected—the pneumatic indicator Pₘᵤₛ is not required for this either,
In order to monitor the patient, the estimated respiratory signal Sig_{res,est} and the respiratory EMG power are determined and output as two vital parameters in a form that can be perceived by a human, preferably visually in the form of a respective time course, optionally together with the airway pressure P_{aw} and/or the esophageal pressure Pₑₛ,
The patient performs his/her own breathing activity and is artificially ventilated for support. A pneumatic indicator Pₘᵤₛ for the patient's own breathing activity is derived using the respiratory signal Sig_{res,est}. The artificial ventilation provided by a ventilator is synchronized as closely as possible with the patient's own breathing activity. In artificial ventilation, the ventilator preferably performs a sequence of ventilation strokes, and in each ventilation stroke a respective quantity of a gas mixture comprising oxygen is delivered to the patient. Preferably, the ventilation strokes of the artificial ventilation are performed depending on the estimated respiratory signal Sig_{res,est}. For example, the ventilator triggers and/or terminates the ventilatory breaths depending on the estimated respiratory signal Sig_{res,est} and/or determines the respective amplitude of each ventilatory breath and/or the time-varying frequency of the ventilatory breaths depending on the estimated respiratory signal Sig_{res,est}. The end of artificial ventilation can also be controlled depending on the estimated respiratory signal Sig_{res,est}.

According to the invention, an intermediate signal is calculated from the sum signal. In the intermediate signal, the influence of the heart activity, i.e. the cardiogenic signal, on the sum signal is approximately compensated for by calculation. This intermediate signal is attenuated according to the invention. The invention is based on the following finding: In an entire heartbeat time period or at least in a segment of the heartbeat time period, the influence of the cardiogenic signal Sigₖₐᵣ on the sum signal is considerably greater, preferably at least 50 times greater, particularly preferably at least 100 times greater, than the influence of the respiratory signal Sigᵣₑₛ. In contrast, the sum signal in the period between two consecutive heartbeat time periods is predominantly or even exclusively determined by the respiratory signal Sigᵣₑₛ. Ideally, the influence of cardiac activity is fully compensated in the intermediate signal, in practice only partially. The attenuation compensates at least approximately for the influence of the cardiogenic signal Sigₖₐᵣ on the intermediate signal that remains after the mathematical compensation.

The use of at least one quality indicator according to the invention takes particular account of the fact that various disturbances can occur during the course of the use phase during the process that the or at least one sum signal sensor measures the respective sum signal. These disturbances can lead to a relatively large cardiogenic signal share remaining in the intermediate signal. This share is reduced depending on the quality indicator generated.

According to the first alternative, the signal processing unit determines a reference attenuation signal segment. Preferably, the signal processing unit determines the reference attenuation signal segment in an initialization phase, with the initialization phase occurring before the use phase. Preferably, the initialization phase and the use phase are performed for the same patient. For the calculation, the signal processing unit uses a sample with several sample elements. Each sample element refers to one heartbeat. Each sample element comprises an intermediate signal segment. This intermediate signal segment is a segment of the intermediate signal and lies in the heartbeat time period of this heartbeat.

For each sample element, the signal processing unit generates a respective power indicator sample element. The power indicator sample element is the time course of an indicator for the electrical power in the heartbeat time period of the heartbeat.

The signal processing unit generates an average power signal segment from the sample elements, namely as a weighted average over the power indicator sample elements. The weighted average comprises several weight factors. These weight factors are calculated using a power quality indicator. The smaller the power quality indicator for a power indicator sample element, the smaller the weight factor for that sample element. The power quality indicator is a quality indicator that describes the shape of the power signal segment of the sample element. It is possible that all weight factors are of the same size, i.e. that an arithmetic average (arithmetic mean) is formed.

The signal processing unit uses the calculated average power signal segment to calculate the reference attenuation signal segment.

In one embodiment, several frequency bands are specified. Several steps just described are performed for each frequency band, preferably in parallel. In particular, the signal processing unit performs the following step for each specified frequency band: It determines a respective component (portion) of the reference attenuation signal segment or determines the component by means of a read access to the data memory. Each component relates to one frequency band.

The signal processing unit also performs the following steps for each specified frequency band and for each heartbeat detected in the use phase:
It generates a component of the intermediate signal segment for the heartbeat time period of the detected heartbeat, with the component occurring in this frequency band.
It generates a component of the attenuated intermediate signal segment for the heartbeat time period, wherein this component occurs in the frequency band. For this purpose, it uses the component of the intermediate signal segment for this frequency band.

The signal processing unit generates the attenuated intermediate signal segment for a heartbeat time period from the components for the frequency bands calculated as just described.

In the first alternative, the signal processing unit performs the following steps:
As just described, the signal processing unit has calculated for each detected heartbeat a component of the intermediate signal segment for the heartbeat. This component relates to the frequency band.
The signal processing unit determines for each frequency band a respective component of the reference attenuation signal segment. This component also relates to the frequency band. To calculate this component, the signal processing unit uses at least one quality indicator.
The signal processing unit applies the component of the reference attenuation signal segment to the component of the intermediate signal segment. This application supplies a component of the attenuated intermediate signal segment.

In the second alternative, the signal processing unit performs the following steps:
The signal processing unit determines for each frequency band a respective component of the reference attenuation signal segment. This component relates to the frequency band.
The signal processing unit determines for each detected heartbeat and for each frequency band a respective component of the adapted attenuation signal segment. This component relates to the frequency band. To calculate this component, the signal processing unit uses at least one quality indicator.
The signal processing unit applies the component of the adapted attenuation signal segment to the component of the intermediate signal segment. The application supplies (yields) a component of the attenuated intermediate signal segment.

The invention further relates to an arrangement or system comprising a signal processing unit according to the invention and at least one sum signal sensor. The arrangement or system may also comprise a display. The or each sum signal sensor of the arrangement is capable of measuring a signal that is generated in and/or on the patient's body. The or each sum signal sensor provides measured values. The signal processing unit receives these measured values and generates at least one sum signal from the received measured values, in one embodiment a sum signal for each sum signal sensor.

The invention is described below by means of embodiment examples. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is an exemplary segment of a cardiogenic signal in the course of a single heartbeat;

FIG. 2 is a diagram that shows which sensors measure which different variables that are used to determine an estimated respiratory signal;

FIG. 3 is a diagram showing an exemplary course of the sum signal as well as two exemplary heartbeat times and four breathing periods;

FIG. 4 is a schematic view of the compensation function block and the attenuation function block;

FIG. 5 is a diagram showing an example of how a cardiogenic reference signal segment is generated under ideal conditions;

FIG. 6 shows diagrams of several correctly positioned segments of the compensation signal with and without interference;

FIG. 7 is a schematic view of the two function blocks of FIG. 4, wherein the attenuation function block is shown in more detail with the compensation function block;

FIG. 8a, FIG. 8b, FIG. 8c, FIG. 8d, and FIG. 8e show diagrams of several exemplary components of an attenuation function;

FIG. 9 is a diagram of average power signal segments Pow_{com,av}(1), . . . , Pow_{com,av}(n) and calculated threshold values φ(1), . . . , φ(n) for the n levels (frequency bands);

FIG. 10 is a diagram of an average power signal segment Pow_{com,av}(5) for level (frequency band) no. 5;

FIG. 11 is a diagram of the reference attenuation signal segments for the n levels;

FIG. 12 is a schematic view showing the attenuation function block of FIG. 4 in more detail;

FIG. 13 is a schematic view showing a measured value processor and the quality indicator function block that determines a quality indicator for the measured value processor;

FIG. 14 is a diagram of a configuration to calculate an average curve (baseline) of the raw signal;

FIG. 15 is a schematic view showing, in detail, the function block that provides two quality indicators;

FIG. 16 shows diagrams of exemplary averaged power signal segments and reference attenuation signal segments;

FIG. 17 is a diagram showing an example of a reference attenuation signal segment and an adapted attenuation signal segment for a heartbeat time period.

### DESCRIPTION OF PREFERRED EMBODIMENTS

Referring to the drawings, in the embodiment examples, the invention is used to automatically determine an estimate signal (a representation signal/indication signal) Sig_{res,est} for a respiratory signal Sigᵣₑₛ, wherein the respiratory signal Sigᵣₑₛ to be estimated correlates with a patient P's own breathing activity and therefore at least approximately describes the patient P's own breathing activity. This patient's own breathing activity can be triggered by electrical impulses in the body of the patient P, wherein the patient P generates these impulses himself/herself, i.e. the patient's breathing activity is a spontaneous respiration, and/or can be stimulated from the outside, for example in a magnetic field. The index est indicates that the respiratory signal Sigᵣₑₛ is estimated, or a representation signal/indication signal, and not measured exactly.

In one application of the embodiment, the patient P is at least temporarily artificially ventilated, namely by supportive artificial ventilation, while the estimated respiratory signal Sig_{res,est} is determined. In another application, the invention is used to monitor the patient P and in particular his/her own breathing activity and to use the respiratory signal Sigᵣₑₛ to be estimated for this purpose, without the patient P necessarily being continuously artificially ventilated.

This respiratory signal Sigᵣₑₛ cannot be measured directly. It is possible to position a measuring probe in the body of patient P and generate measured values from the probe. It is also possible to obtain measured values by non-invasive means, in particular by using electrodes (of one or more sum signal sensors) on the skin of patient P to record measured values. As a rule, it is not possible, neither invasively nor non-invasively, to directly measure the impulses generated in patient P's body wherein these impulses “control” the respiratory muscles, but only electrical measured values that are generated when the muscle fibers of the respiratory muscles contract, or the effects of such electrical measured values on a pneumatic signal, for example. In addition, the electrical impulses that cause patient P's own breathing activity are superimposed by electrical impulses that cause patient P's cardiac activity, or more precisely: that cause the heart muscles to contract. Therefore, only a sum signal Sig_{Sum} can be measured directly after appropriate processing of the measured values. This sum signal Sig_{Sum} results from a superposition of the respiratory signal Sigᵣₑₛ, which correlates with the breathing activity of the patient P, and a cardiogenic signal Sigₖₐᵣ, which correlates with his/her cardiac activity. The sum signal Sig_{Sum} can be influenced by other signals, in particular by signals that affect a transmission channel from the signal source in the patient's body to a measurement location, as well as by external signal sources. These other signals are usually disturbance variables. The measured values from which the sum signal Sig_{Sum} is generated are measured at this measuring location.

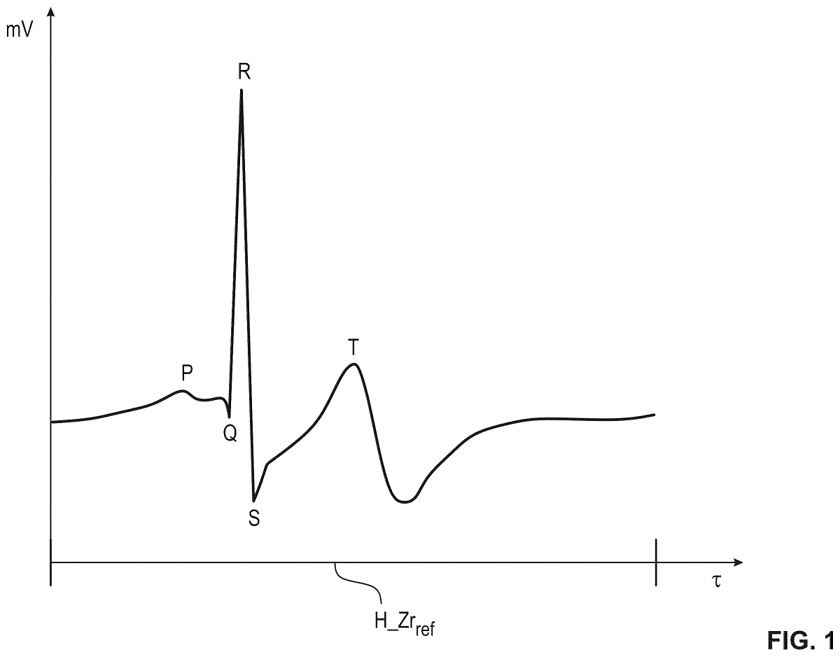

FIG. 1 shows a typical segment of an electrically measured cardiogenic signal Sigₖₐᵣ over the course of a single heartbeat. As an example, a reference heartbeat time period H_Zr_{ref} is shown on the x-axis and the signal value, for example in millivolts, on the y-axis. Five peaks P, Q, R, S, and T can be seen. A characteristic heartbeat time point is, for example, the Q peak, the R peak, the S peak, or also the midpoint in time between the Q peak and the S peak of this heartbeat or the midpoint in time between the P peak and the T peak.

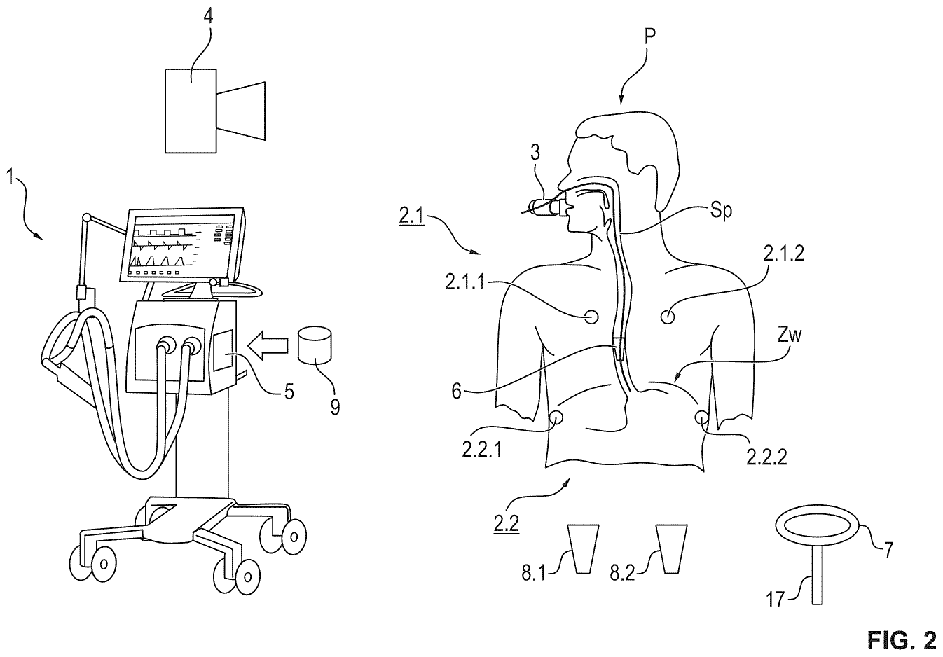

FIG. 2 shows schematically which signals can be generated from measured values by generating the measured values on and/or in the body of the patient P and processing them automatically in a suitable manner. The following entities are shown schematically:
the artificially ventilated patient P,
the esophagus Sp and the diaphragm Zw of the patient P,
a ventilator 1 which artificially ventilates the patient P at least temporarily and which comprises a signal processing unit 5, wherein the signal processing unit 5 has at least temporary read access and write access to a data memory 9,
an intercostal pair 2.1 with two measuring electrodes 2.1.1 and 2.1.2, which are arranged to the right and left of the sternum and between two ribs of the patient P, i.e. in an area close to the heart,
a pair 2.2 close to the diaphragm with two further measuring electrodes 2.2.1 and 2.2.2, which are arranged close to the diaphragm Zw of patient P, i.e. in an area remote from the heart,
an electrode for ground, not shown,
a pneumatic sensor 3, which is spatially remote from the body of patient P and comprises a measured value transducer, which is arranged, for example, in front of the mouth of patient P, and a data-processing evaluation unit, which can be arranged in the ventilator 1,
an optional optical sensor 4, which comprises an image recording device and an image evaluation unit and is directed towards the body of the patient P,
an optional pneumatic sensor 6 in the form of a probe or a balloon in the esophagus Sp and near the diaphragm Zw of the patient P,
a schematically shown cuff 7 around a wrist of the patient P, this cuff 7 holding a catheter 17 in order to invasively measure the time course of the blood pressure,
two finger clips 8.1, 8.2, each placed over a finger of the patient P or positioned elsewhere on the skin of the patient P, one finger clip 8.1 non-invasively measuring the degree of saturation of the blood with oxygen, preferably by means of a plethysmographic process, and the other finger clip 8.2 non-invasively measuring the blood pressure of the patient P,
optionally not shown electrodes in the patient's esophagus P, and
a display unit on which the time courses of signals are displayed.

The intercostal pair 2.1 and the ground electrode provide a first sum signal Sig_{Sum}(1) after signal conditioning (signal processing). Pair 2.2 near the diaphragm and the ground electrode provide a second sum signal Sig_{Sum}(2) after signal conditioning. The other sensors described above can provide further sum signals Sig_{Sum}(n), n>=3. It is also possible for the same sensor arrangement to provide two different sum signals, for example by using different measurement processes. Such a sensor arrangement is described, for example, in DE 10 2009 035 018 A1 and US 2011/0 028 819 A1 (US 2011/0 028 819 A1 is incorporated herein by reference). In the following, the term “the sum signal Sig_{Sum}” is used for short.

Instead of an electrical signal (EMG signal), a sum signal Sig_{Sum} in the form of a mechanomyogram (MMG signal) can also be generated and used.

In order to control the ventilator 1 during artificial ventilation of the patient P or to monitor the patient P and to use the estimated respiratory signal Sig_{res,est} for the control or monitoring (via a visualizing display shown in FIG. 1), the estimated respiratory signal Sig_{res,est} is determined at a high sampling frequency, i.e. at each sampling time t the signal processing unit 5 supplies a new signal value Sig_{res,est}(t). A “high sampling frequency” is understood to mean that there is an interval of less than five milliseconds, preferably less than three milliseconds, between two successive sampling time points. In particular for fatigue determination, the sampling frequency is preferably at least 1 kHz, particularly preferably at least 2 kHz. However, some steps of the process described below are carried out in the embodiment example with a low sampling frequency, namely with a frequency that is in the range of the heartbeat frequency, i.e. between 1 Hz and 2 Hz.

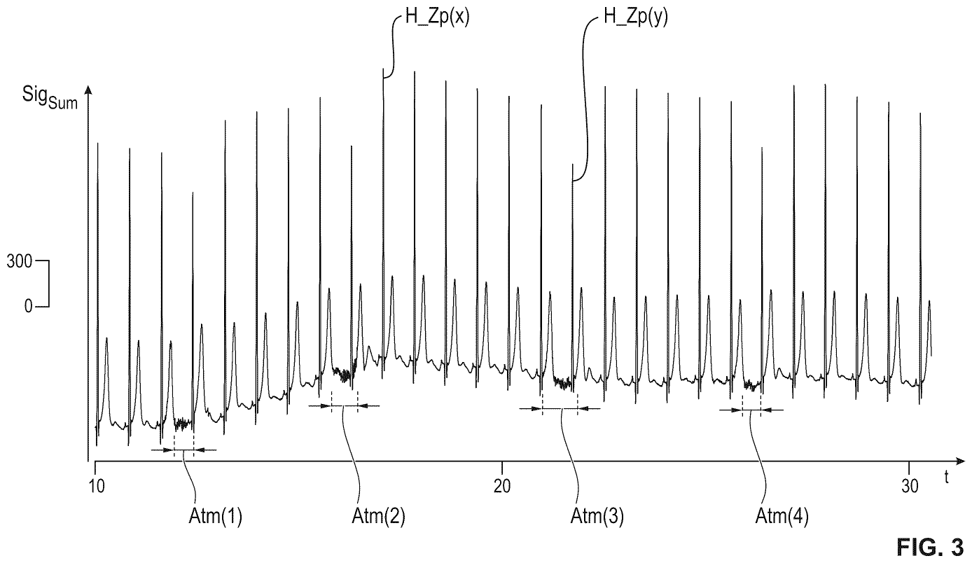

FIG. 3 shows an exemplary time course of the sum signal Sig_{Sum} with four breaths and a large number of heartbeats. The time is plotted on the x-axis and a variable measured by the sum signal sensor, for example an electrical voltage in millivolts, is plotted on the y-axis. The four time durations (time periods) Atm(1), . . . , Atm(4) of the four breaths and, for example, two characteristic heartbeat times H_Zp(x) and H_Zp(y) are shown. It can be seen that the cardiogenic signal Sigₖₐᵣ in a heartbeat time period H_Zp(x), H_Zp(y) is many times greater than the respiratory signal Sigᵣₑₛ in this time period. Outside a heartbeat time period, however, the respiratory signal Sigᵣₑₛ is sufficiently strong compared to the cardiogenic signal Sigₖₐᵣ and can therefore be determined from the sum signal Sig_{Sum}.

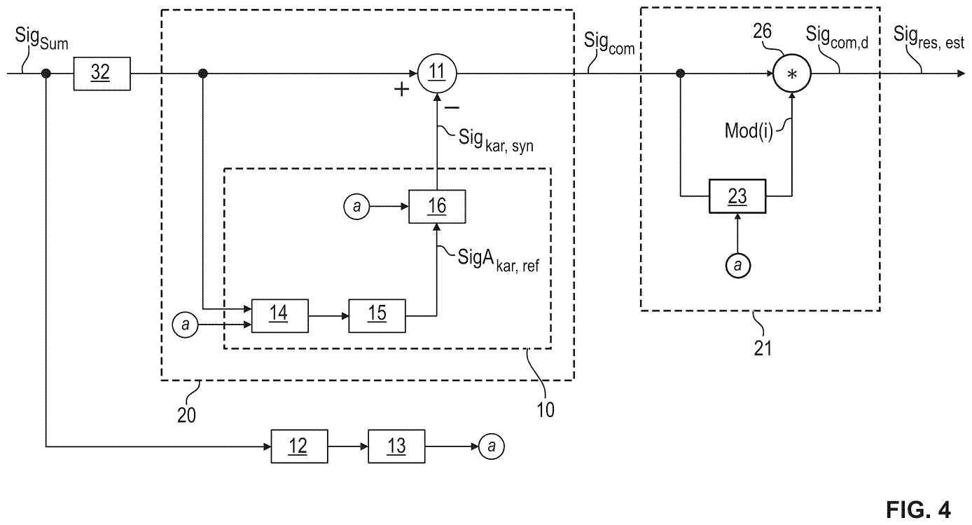

FIG. 4 schematically shows two function blocks 20 and 21 of the signal processing unit 5, wherein the function blocks 20, 21 each perform different signal processing steps in order to at least partially compensate for the influence of the cardiac activity Sigₖₐᵣ on the measured sum signal Sig_{Sum}. The output signal of a compensation function block 20, namely a compensation signal Sig_{com} described below, is applied as an input signal to an attenuation function block 21. The attenuation function block 21 supplies the required estimate Sig_{res,est} as the output signal.

A functional unit 10 of the compensation function block 20 generates a synthetic cardiogenic signal Sig_{kar,syn}, which is an approximation (estimate) for the cardiogenic signal Sigₖₐᵣ and is composed of signal segments (hence the term synthetic). Each signal segment describes the cardiac activity in the course of a heartbeat. An example of such a signal segment is shown in FIG. 1. The signal segments are positioned correctly in time (correctly timed), optionally adapted, and combined to form the synthetic cardiogenic signal Sig_{kar,syn}. The synthetic cardiogenic signal Sig_{kar,syn} is an estimate of the actual cardiogenic signal Sigₖₐᵣ. A process for adapting a signal segment is described in DE 10 2019 006 866 A1 and US 2022/0 330 837 A1 (US 2022/0 330 837 A1 is incorporated herein by reference).

The compensation function block 20 computationally compensates, for example by subtraction, the contribution of the synthetic cardiogenic signal Sig_{kar,syn} to the sum signal Sig_{Sum} and thereby generates the compensation signal Sig_{com} serving as the intermediate signal.

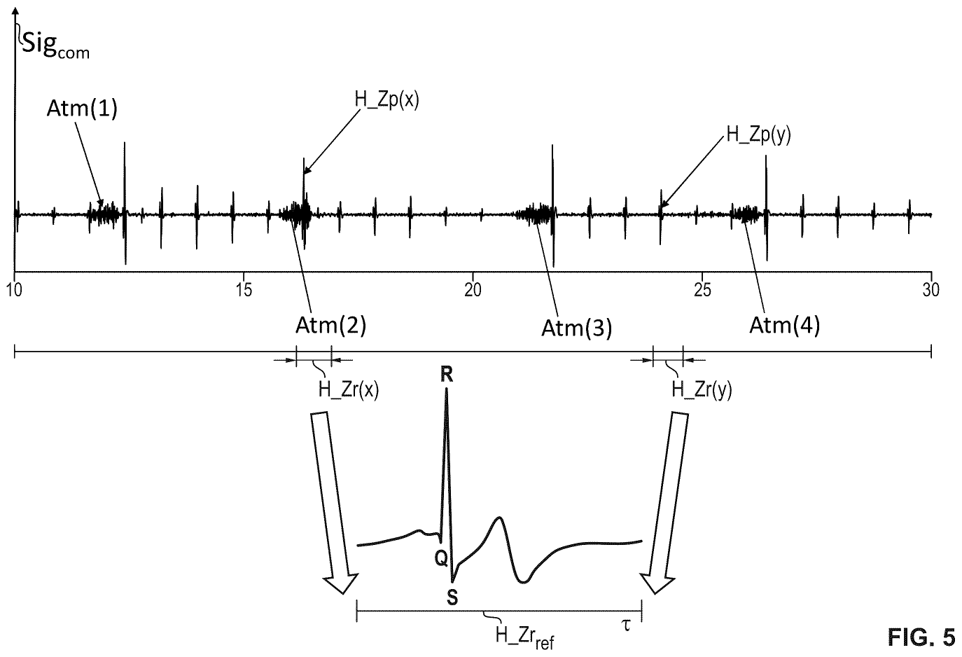

FIG. 5 shows an example time course of the compensation signal Sig_{com}. This exemplary time course results from the fact that the compensation function block 20 processes the sum signal Sig_{Sum} shown as an example in FIG. 3, as just described. FIG. 5 also shows two exemplary heartbeat time periods H_Zp(x) and H_Zp(y). It is illustrated how these two heartbeat time periods H_Zp(x) and H_Zp(y) are mapped to the same reference heartbeat time period H_Zr_{ref}.

In an initialization phase, the compensation function block 20 of FIG. 4 generates a cardiogenic reference signal segment SigA_{kar,ref}, which is stored in the data memory 9, and reapplies it in a subsequent use phase for each detected heartbeat. The following steps are performed:
A functional unit 12 identifies the respective start and end and/or the respective QRS phase of each heartbeat in the sum signal Sig_{Sum}, i.e. the respective characteristic heartbeat time period H_Zr(x), H_Zr(y).
A functional unit 13 determines the respective exact characteristic heartbeat time point H_Zp(x), H_Zp(y) of each heartbeat, preferably with a tolerance of a few milliseconds. Particularly preferably, the tolerance is at most half the time duration between two successive sampling times for determining the sum signal Sig_{Sum}, this time duration preferably being less than 1 millisecond.
In order to determine the exact heartbeat time H_Zp(x) of each heartbeat, the functional units 12 and 13 require several values of the sum signal Sig_{Sum} for several successive sampling time points. In one embodiment, an optional delay functional unit 32 delays the sum signal Sig_{Sum} for the subsequent steps by a corresponding time period, see FIG. 4. As a result, the exact heartbeat time point H_Zp(x) is available in the subsequent steps. This optional functional unit 32 is omitted in the following figures. This delay is only used if the respective application does not require the estimated respiratory signal Sig_{res,est} in real time.

In the initialization phase, N heartbeats are detected. Each heartbeat no. x has a segment SigA_{Sum}(x) of the sum signal Sig_{Sum}. These N heartbeats therefore provide N sample elements for a sample.

In the initialization phase, the following steps are also carried out:
A functional unit 14 computationally superimposes the N sum signal segments SigA_{Sum}(x₁), . . . , SigA_{Sum}(x_{N}) for the last N heartbeats x₁, . . . , x_{N}, with the correct time. If necessary, these N sum signal segments are cut to a matching (coinciding) length or compressed or stretched.
Preferably, the signal segments SigA_{Sum}(x₁), . . . , SigA_{Sum}(x_{N}) for the N heartbeats are superimposed in such a way that they have the same length, and the R peaks or other characteristic heartbeat times lie on top of each other. Each signal segment thus refers to the same reference heartbeat time period H_Zr_{ref}, see FIG. 5. A relative time point in this reference heartbeat time period H_Zr_{ref} is denoted by T. Each absolute time point t of the sum signal segment SigA_{Sum}(x) corresponds to a relative time point τ=τ(t) in this relative heartbeat time period. Instead of the term “relative time point”, the term “heart phase φ” with a value range from 0° to 360° or from 0 to 2π can also be used.
A functional unit 15 generates a cardiogenic reference signal segment (template) SigA_{kar,ref} from the superposition of N signal segments SigA_{Sum}(x₁), . . . , SigA_{Sum}(x_{N}) generated by the functional unit 14. This cardiogenic reference signal segment SigA_{kar,ref} approximately describes the course of the cardiogenic signal Sigₖₐᵣ during a single heartbeat and also refers to the reference heartbeat time period H_Zr_{ref}. Preferably, the characteristic heartbeat time point H_Zp(x) is at τ=0. As already mentioned, during a heartbeat, the cardiogenic component in the sum signal Sig_{Sum} is many times greater than the respiratory component, and by averaging over N signal segments, the respiratory components during a heartbeat time period are largely “averaged out”.
Preferably the functional unit 15 applies a learning process to the N signal segments for the respective last N heartbeats. The cardiogenic reference signal segment SigA_{kar,ref} is preferably stored in the data memory 9. This cardiogenic reference signal segment SigA_{kar,ref} refers to the patient P and his/her current condition, i.e. it is not an averaging of signals from different patients.

The following steps are carried out in a subsequent use phase:
The functional units 32 and 13 detect the heartbeats in the sum signal Sig_{Sum} and determine the respective characteristic heartbeat time of each detected heartbeat.
For each heartbeat no. x, the cardiogenic reference signal segment SigA_{kar,ref} is used again. In one embodiment, this is subtracted unchanged from the sum signal segment SigA_{Sum}(x) for the heartbeat time period H_Zr(x) of the heartbeat x (template subtraction).
Optionally, however, a functional unit 16 uses the value of at least one anthropological parameter which influences the cardiac activity and thus the cardiogenic signal Sigₖₐᵣ and which has been measured during this heartbeat no. x. The lung filling level and an indicator of the patient's current posture P as well as the distance RR between the R peaks of two consecutive heartbeats are examples of such an anthropological parameter. For each heartbeat, the functional unit 16 adjusts the cardiogenic reference signal segment SigA_{kar,ref} to the or each anthropological parameter value measured during this heartbeat, thereby generating a cardiogenic signal segment SigAₖₐᵣ(x). Such a procedure is described, for example, in DE 10 2019 006 866 A1 (US 2022/0 330 837 A1) and DE 10 2020 002 572 A1 (US 2021/0 338 176 A1) (US 2021/0 338 176 A1 is incorporated herein by reference).
The functional unit 16 positions the cardiogenic reference signal segment SigA_{kar,ref} or optionally the adapted cardiogenic signal segment SigAₖₐᵣ(x) relative to the sum signal segment SigA_{Sum}(x) of the current heartbeat no. x in the correct time, e.g. QRS-synchronized. This generates a new synchronized segment SigA_{kar,syn}(x) of the synthetic cardiogenic signal Sig_{kar,syn}. Preferably, the synthetic cardiogenic signal Sig_{kar,syn} is output in a form that can be perceived by a human, such as on the visualizing display.
A functional unit 11 compensates for the influence of the cardiogenic signal Sigₖₐᵣ in the most recent sum signal segment SigA_{Sum}(x), for example by the functional unit 11 subtracting the cardiogenic reference signal segment SigA_{kar,ref} or the adapted cardiogenic signal segment SigAₖₐᵣ(x) from the latest (most recent) sum signal segment SigA_{Sum}(x). The result is a new segment SigA_{com}(x) of the compensation signal Sig_{com}.

At the beginning of the procedure, i.e. after the patient P is connected to the measuring electrodes 2.1.1 to 2.2.2, the initialization phase is carried out, which comprises a period of N detected heartbeats. In this initialization phase, the compensation function block 20 generates an initial cardiogenic reference signal segment SigA_{kar,ref} for the last N heartbeats depending on the sum signal segments SigA_{Sum}(x₁), . . . , SigA_{Sum}(x_{N}) as described above. During the procedure, the compensation function block 20 adapts the cardiogenic reference signal segment SigA_{kar,ref} preferably to the respective last N heartbeats and stores the result in the data memory 9. The steps in the initialization phase and the adaptation to the last N heartbeats are performed at the low sampling frequency, which is approximately equal to the heartbeat frequency.

Preferably, the segments for a heartbeat are superimposed with twice the time resolution of the sum signal Sig_{Sum}. This means: The values of the sum signal Sig_{Sum} are determined with a high sampling frequency f, i.e. the interval Δt between two sampling times is 1/f The time resolution is increased computationally to e.g. 2f or 3f, e.g. by computationally positioning a signal value Sig_{Sum}(t) and Sig_{Sum}(t+Δt) between two signal values Sig_{Sum}(t+Δt/2) derived from measured values, for example by interpolation.

After the initialization phase, the following steps are carried out at a high sampling frequency (a few milliseconds or even just a few tenths of a millisecond):
The signal processing unit 5 derives a new value Sig_{Sum}(t) for the sum signal Sig_{Sum} from the measured values.
The functional units 12 and 13 recognize the start of the heartbeat time period H_Zr(x) or the exact characteristic time H_Zp(x) of a heartbeat x, resp., in the sum signal Sig_{Sum} and thereby determine a new sum signal segment SigA_{Sum}(x).
The compensation function block 20 optionally adapts the cardiogenic reference signal segment SigA_{kar,ref} to the respective value of at least one anthropological parameter. The compensation function block 20 determines the assigned relative time point τ=τ(t) and generates a further signal segment, namely the most recent segment SigA_{kar,syn}(x) of the synthetic cardiogenic signal Sig_{kar,syn}, by positioning it correctly in terms of time.
The functional unit 11 subtracts from the new value Sig_{Sum}(t) the value SigA_{kar,ref}[τ(t)] or SigAₖₐᵣ(x) [τ(t)] of the cardiogenic reference signal segment SigA_{kar,ref} or of the adapted cardiogenic signal segment SigAₖₐᵣ(x) for the same relative time point τ, i.e.

$$S i {g}_{c o m} ( t ) = {Sig}_{Sum} ( t ) - S i g {A}_{kar , syn} [ τ ( t ) ]$$

or compensates for the cardiogenic influence in some other way.
The compensation function block 20 outputs a new signal segment SigA_{com}(x) for the compensation signal Sig_{com}.

Ideally, the compensation signal Sig_{com} contains all contributions of the cardiac activity Sigₖₐᵣ to the sum signal Sig_{Sum}. In practice, this is not the case. There are two main possible causes for this
Possible disturbances in the initialization phase lead to a cardiogenic reference signal segment SigA_{kar,ref}, which deviates from reality in a relevant way.
Possible faults in the use phase influence the sum signal Sig_{Sum}.

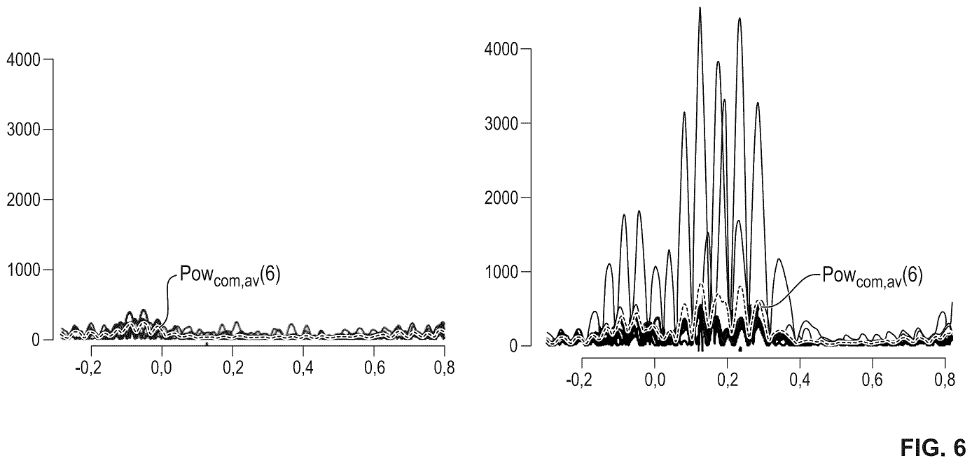

FIG. 6 illustrates the effects of possible interference. In both diagrams, several signal segments are shown for the time course of an electrical power of the signal, including the signal segment Pow_{com,av}(6), which is explained below. Each signal segment covers a single heartbeat time period H_Zr(x), H_Zr(y). In the left-hand diagram, no disturbances occur, while in the right-hand diagram several disturbances occur that lead to large oscillations of the compensation signal Sig_{com}. These disturbances occurred, for example, when the measured values were generated. Without suitable countermeasures, these large oscillations can lead to incorrect results.

In the embodiment example, an attenuation function block 21 is used to post-process the compensation signal Sig_{com}, see FIG. 4. The output signal of the compensation function block 20, namely the compensation signal Sig_{com}, is present as an input signal at the attenuation function block 21.

A functional unit 23 of the attenuation function block 21 generates from the compensation signal Sig_{com} an attenuation signal segment Mod(i) described below. A functional unit 26 applies this attenuation signal segment Mod(i) to the compensation signal Sig_{com}, thereby computationally causing a reduction in the electrical power, in particular an attenuation, and thereby generating the estimated respiratory signal Sig_{res,est}.

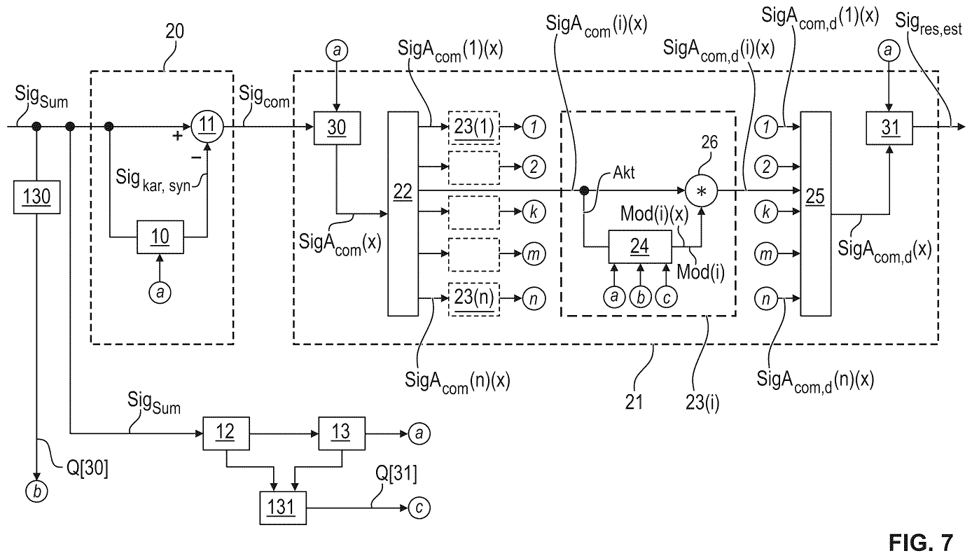

The attenuation function block 21 is described in more detail below with reference to FIG. 7. The compensation signal Sig_{com} is applied to the attenuation function block 21.

N frequency bands are specified, which are also called “levels” in a wavelet transformation. Here, n is a predefined number. Preferably, n is between 5 and 10 and is particularly preferably 8. Level 1 belongs to the frequency band with the highest frequencies, level n belongs to the frequency band with the lowest frequencies.

Unless otherwise stated, the following description refers to the use phase.

A functional unit 30 generates the compensation signal segment SigA_{com}(x) for the latest (most recently) detected heartbeat x from the compensation signal Sig_{com}. For this purpose, it uses the characteristic heartbeat time point H_Zp(x) and the heartbeat time period H_Zr(x), which the functional units 12 and 13 have detected using the sum signal Sig_{Sum}.

A functional unit 22 decomposes (breaks down/deconstructs) the compensation signal segment SigA_{com}(x) of the compensation signal Sig_{com} into n signal component segments SigA_{com}(1)(x), . . . , SigA_{com}(n)(x) for the n levels (frequency bands). Preferably, the functional unit 22 performs a wavelet transformation, preferably a stationary wavelet transformation or a transformation à trous. If the signal component segments SigA_{com}(i)(x) are joined together (combined) correctly in time, a signal component Sig_{com}(i) (i=1, . . . , n) is produced.

The attenuation function block 21 comprises the functional unit 22 for the decomposition, a functional unit 25 for the reverse transformation (back-transformation) and, for each level i, a respective functional unit 23(i) and two functional units 24=24(i) and 26=26(i). In FIG. 7, only one functional unit 24 and one functional unit 26 are shown, namely for level i.

In the initialization phase, the reference attenuation signal segment functional unit 23(i) generates a reference attenuation signal segment Mod(i) for each level i (i=1, . . . , n), i.e. a total of n reference attenuation signal segments Mod(1), . . . , Mod(n). Each reference attenuation signal segment Mod(i) describes a time course and covers the reference heartbeat time period H_Zr_{ref}. Each signal value Mod(i)(τ) is a number between 0 and 1 (inclusive). For each level i, in the initialization phase a respective reference attenuation signal segment Mod(i) is therefore generated. These n reference attenuation signal segments Mod(1), . . . , Mod(n) are stored in the data memory 9 and used in the use phase

A respective reference attenuation signal segment Mod(i) is therefore generated for each level i in the initialization phase. The arrow Akt in block 23(i) in FIG. 7 indicates that, in one embodiment, the reference attenuation signal segment Mod(i) is also continuously updated in the use phase. How this is done is described below.

In the use phase, the respective functional unit 23(i) is applied to the signal component segment SigA_{com}(i)(x) for the heartbeat x and for each level i, i=1, . . . , n. The functional unit 24=24(i) of the functional unit 23(i) generates an adapted attenuation signal segment Mod(i)(x) from the reference attenuation signal segment Mod(i), wherein the adapted attenuation signal segment Mod(i)(x) represents a time course and covers the heartbeat time period H_Zr(x) and wherein each signal value Mod(i)(x)(t) is a number between 0 and 1 (inclusive).

In the use phase, a functional unit 26=26(i) applies the correctly positioned in time adapted attenuation signal segment Mod(i)(x) to the signal component segment SigA_{com}(i)(x) for the heartbeat x and generates the attenuated signal component segment SigA_{com,d}(i)(x) (i=1, . . . , n). For example, the functional unit 26 multiplies the two signal values SigA_{com}(i)(x)(t) and Mod(i)(x)[τ(t)] with each other and thereby generates a value SigA_{com,d}(i)(x)(t) of the attenuated signal component segment SigA_{com,d}(i)(x) for each sampling time t, for example according to the calculation rule

$$S i g {A}_{c o m , d} ( i ) ( x ) ( t ) = S i g {A}_{c o m} ( i ) ( x ) ( t ) * Mod ( i ) ( x ) ( t ) .$$

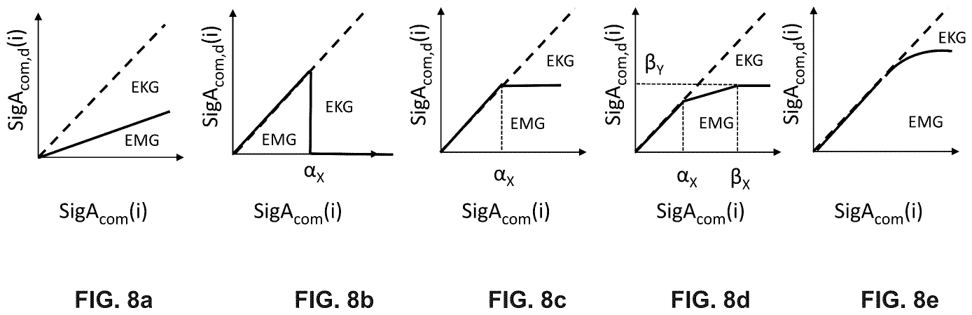

Further possible implementations are described below with reference to FIG. 8.

This modification provides an attenuation SigA_{com,d}(i)(x) of the signal component segment SigA_{com}(i)(x). The sign of each signal value is retained during attenuation. Alternative embodiments of the attenuation are described below.

FIGS. 8a, 8b. 8c. 8d, and 8e illustrate five alternative ways in which the attenuated signal component segment SigA_{com,d}(i)(x) is generated by attenuation from the signal component segment SigA_{com}(i)(x). The attenuation splits the signal component segment SigA_{com}(i)(x) into a respiratory component SigA_{com,d}(i), which is also referred to as EMG in FIGS. 8a-e, and a cardiogenic component, which is referred to as EKG (ECG).

Option a) (FIG. 8a) is the configuration just described, multiplied by a factor Mod(i)(x), wherein the slope Mod(i)(x)(t) of the straight line depends on t. Option b) (FIG. 8b) means a hard threshold value α, wherein this threshold value α=α(t) also depends on t. Option c) (FIG. 8c) means a soft threshold. Option d) (FIG. 8d) is a mixed form. Option e) (FIG. 8e) is described below.

Therefore, the attenuation generates an attenuated signal component segment SigA_{com,d}(i)(x), which relates to the time period H_Zr(x) of the last heartbeat no. x and to level no. i.

The functional unit 25 combines the attenuated signal component segments SigA_{com,d}(1)(x), . . . , SigA_{com,d}(n)(x) to form an attenuated signal component segment SigA_{com,d}(x), wherein the functional unit 25 preferably performs a wavelet reverse transformation, and outputs this attenuated signal component segment SigA_{com,d}(x) as an output signal.

The functional unit 31 generates the sought estimated respiratory signal Sig_{res,est}. For this purpose, it uses the characteristic heartbeat times H_Zp(x), the heartbeat time periods H_Zr(x), and the attenuated signal component segments SigA_{com,d}(x). For a segment that lies between two consecutive heartbeat time periods H_Zr(x) and H_Zr(x+1), the functional unit 31 preferably uses the corresponding segment of the compensation signal Sig_{com} as the segment of the estimated respiratory signal Sig_{res,est}. The functional unit 31 outputs the estimated respiratory signal Sig_{res,est} generated in this manner.

The functional units 14 and 15 shown in FIG. 4 update the reference cardiogenic signal segment SigA_{kar,ref} as soon as another (further) heartbeat is completed, i.e. they generate an adapted cardiogenic signal segment SigAₖₐᵣ(x). In addition, the functional unit 23(i) adjusts the reference attenuation signal segments Mod(i) for the n levels and thereby generates the adapted attenuation signal segment Mod(i)(x), preferably as soon as the further heartbeat is completed (i=1, . . . , n).

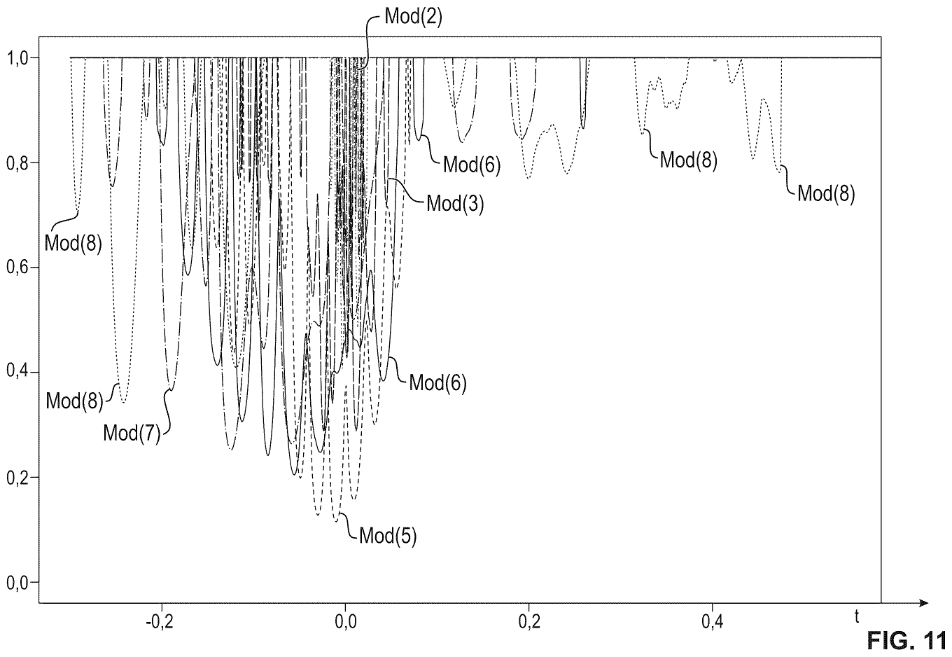

The following describes how the n reference attenuation signal segments Mod(1), . . . , Mod(n) are generated in the initialization phase. FIG. 11 shows an example of eight reference attenuation signal segments Mod(1), . . . , Mod(8), i.e. n=8. In one embodiment, the functional unit 24 performs in the initialization phase the steps described below for each level i and for each signal component segment SigA_{com}(i)(x) of a heartbeat x (i=1, . . . , n):

In the initialization phase, the functional unit 24 determines an average power signal segment Pow_{com,av}(i) for the time course of an electrical power, the power signal segment Pow_{com,av}(i) covering the reference heartbeat time period H_Zr_{ref} and relating to level no. i. The average power signal component Pow_{com,av}(i) is calculated as a weighted average over the power values of the M signal component components SigA_{com}(i)(x) of M heartbeats x. For example

$$P o {w}_{c o m} ( i ) ( x ) ( τ ) = Abs [ S i g {A}_{c o m} ( i ) ( τ ) ] ( the absolute value ) or$$

$$Po {w}_{c o m} ( i ) ( x ) ( τ ) = RMS [ {SigA}_{c o m} ( i ) ( τ ) ] ( root mean square , RMS , the effective value ) .$$

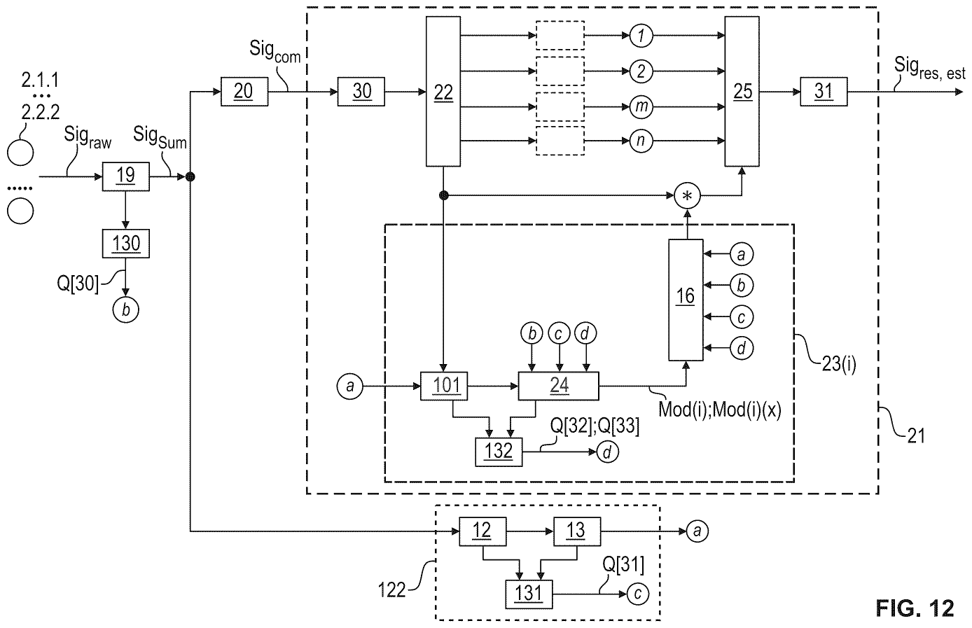

FIG. 6 shows an example of the average signal segment Pow_{com,av}(6) for level No. 6, which was calculated as an arithmetic average. Further below, with reference to FIG. 12, it is explained how the functional unit 24 forms the weight factor for the weighted averages.

In the initialization phase, M power signal segments Pow_{com}(i)(x) are by this calculated for the M heartbeats. The numbers M and N (number of heartbeats for calculating the cardiogenic reference signal segment SigA_{kar,ref}) can be the same or different from each other. Preferably, each power signal component Pow_{com}(i)(x) is calculated using a suitable filter, with suitable smoothing over values of the compensation signal Sig_{com}.

Each power signal segment Pow_{com}(i)(x) of a heartbeat x covers a heartbeat time period H_Zr(x). The functional unit 24 superimposes the M power signal segments Pow_{com}(i)(x) on the M heartbeats synchronously (in time) and then forms a weighted average over the superimposed M segments. This determines an average power signal segment Pow_{com,av}(i) for level no. i, which is an indicator of the average electrical power of the compensation signal Sig_{com}(i) in level no. i during the reference heartbeat time period H_Zr_{ref}, wherein the determined average electrical power depends on the relative time T. Averaging “averages out” influencing factors that are not caused by the cardiac activity of the patient P, but by the breathing activity, for example by a cough.

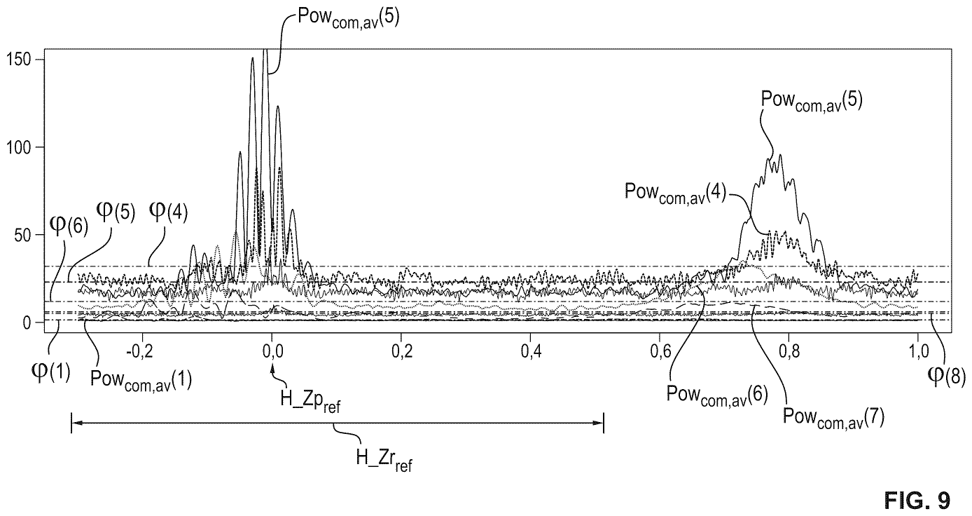

FIG. 9 shows the reference heartbeat time period H_Zr_{ref} and the reference heartbeat time point H_Zp_{ref} of this average power signal segment Pow_{com,av}(i) generated by heartbeat-synchronous superposition. In this example, eight different levels are distinguished, i.e. n=8. The time point t=0 on the x-axis was placed at the reference heartbeat time point H_Zr(_{ref}). Furthermore, FIG. 9 shows the n=8 average power signal segments Pow_{com,av}(1), . . . , Pow_{com,av}(8) for the n=8 levels.

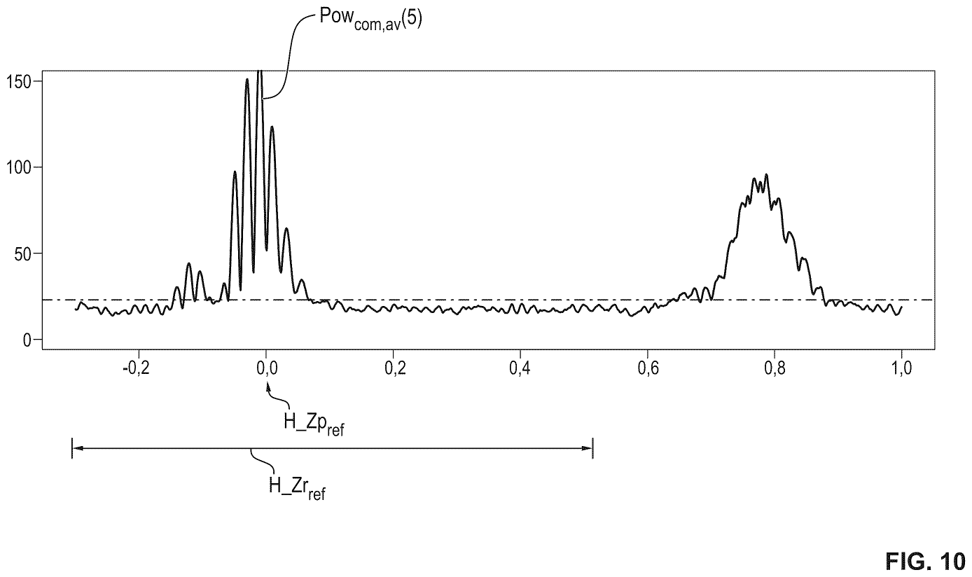

FIG. 10 shows the average power signal segment Pow_{com,av}(5) for level no. 5.

An average signal value Avg(i) is derived from the average power signal segment Pow_{com,av}(i) for level no. i. Furthermore, a threshold value φ(i) is derived using the average power signal segment Pow_{com,av}(i) and the average signal value Avg(i). The average signal value Avg(i) and the threshold value φ(i) usually vary from level i1 to level i2 and also for a single level i from heartbeat to heartbeat if the average signal value Avg(i) and the threshold value φ(i) are continuously updated depending on the last (most recent) M heartbeats. With the aid of this threshold value φ(i), which depends on the compensation signal Sig_{com}, noise in the compensation signal Sig_{com} is later at least partially eliminated by computation, wherein this noise is essentially generated by the cardiogenic signal Sigₖₐᵣ. Thanks to the procedure just described, the threshold values φ(1), . . . , φ(n) are calculated at runtime and do not need to be specified.

The average signal value Avg(i) is calculated, for example, as an arithmetic average (arithmetic mean) or also as a median over R signal values of the average power signal segment Pow_{com,av}(i) at R consecutive relative sampling times τ₁, . . . , τ_{R} of the reference heartbeat time H_Zr_{ref}. The median is less sensitive to outliers (freak values) than the arithmetic average, but its calculation requires more computing time.

To calculate the threshold value φ(i), a factor α is specified, for example α=2. The threshold value φ(i) is calculated, for example, according to the calculation rule

$$φ ( i ) = [ 1 + ( n - i ) / α * n ] * Av g ( i ) .$$

FIG. 9 also shows the n threshold values φ(1), . . . , φ(n) for the n levels.

A signal value SigA_{com}(i)(x)(t) of the signal component SigA_{com}(i)(x) of the compensation signal Sig_{com} should be attenuated to a greater extent, the larger (the higher) the signal value Pow_{com,av}(i)(τ) of the average power signal component Pow_{com,av}(i) is at the corresponding relative time τ(t) of the reference heartbeat time period H_Zr_{ref}. This is because—due to averaging over N heartbeat time periods—large signal values originate from the cardiogenic signal Sigₖₐᵣ. The attenuation therefore depends on the currently determined sum signal Sig_{Sum} and not on a predefined threshold value. As already mentioned, the attenuation according to this embodiment also depends on the relative time point T during a reference heartbeat time period H_Zr_{ref}. In this way, the attenuation can be adapted to the current cardiac activity of the patient P, even in the event of irregularities in the cardiac activity.

In one embodiment, a reference attenuation signal segment Mod(i) is generated from the average power signal segment Pow_{com,av}(i), for example according to the following calculation rule:

$$Mod ( i ) ( τ ) = min \{ Av g ( i ) / P o {w}_{com , av} ( i ) ( τ ) , 1 \} ,$$

if τ is in the reference heartbeat time period H_Zr_{ref} and Pow_{com,av}(i)(τ)>φ(i) applies, and Mod(i)(τ)=1 otherwise.
   Each signal value Mod(i)(τ) of the reference attenuation signal segment Mod(i) is a number between 0 and 1 (inclusive).

The configuration in which the signal value Mod(i)(τ) is set to 1 outside the reference heartbeat time period H_Zr_{ref} ensures that the reference attenuation signal segment Mod(i) only causes attenuation for the current heartbeat.

In a generalization, each value for Mod(i) is calculated according to the calculation rule

$$Mod ( i ) ( τ ) = min \{ F [ P o {w}_{com , av} ( i ) ( τ ) ] , 1 \}$$

where F=F(u) is a function decreasing in u [the larger u, the smaller F(u)] and has a value range from 0 to y and where y is greater than or equal to 1.

As shown in FIG. 8, there are alternatives to the embodiment of achieving the attenuation by multiplication. In several embodiments, which are shown in FIG. 8 b) to FIG. 8 d), a threshold value α_{X}=α_{X}(τ) is used. In one embodiment, which is shown in FIG. 8 d), two additional threshold values β_{X}=β_{X}(τ) and β_{Y}=β_{Y}(τ) are also used.

FIG. 12 shows an extension of the functional circuit diagram of FIG. 7 in accordance with the invention. The same reference signs have the same meanings as in FIG. 7.

FIG. 12 schematically shows a measured value processor 19. This measured value processor 19 processes the raw signal Sig_{raw}, which is supplied by the sensors 2.1.1 to 2.2.2 after signal amplification. The measured value processor 19 computationally removes low-frequency oscillations, normalizes the raw signal Sig_{raw}, and supplies the sum signal Sig_{Sum}.

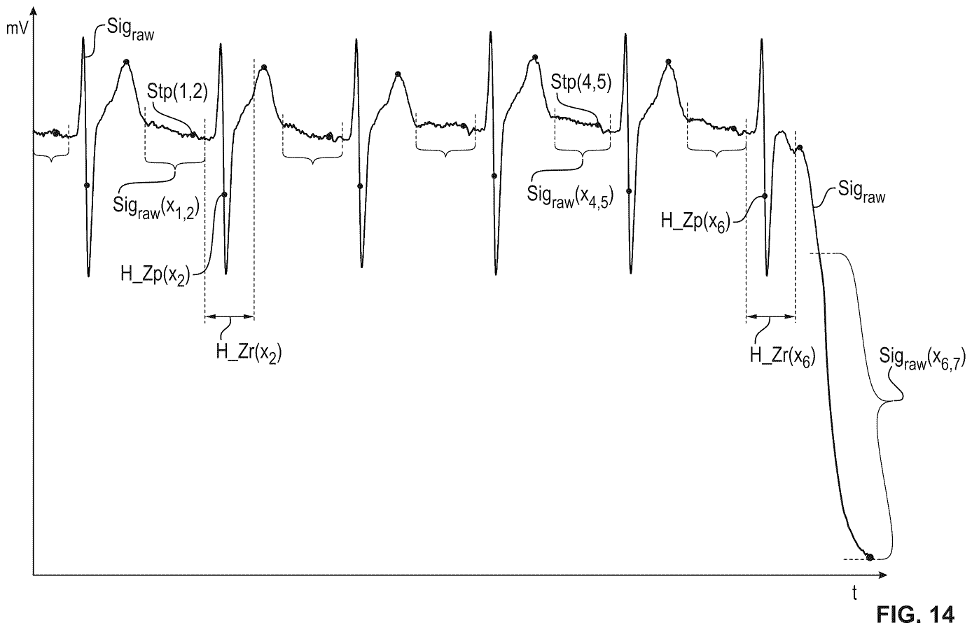

In one embodiment, the measured value processor 19 subtracts a type of average curve (baseline) BL from the raw signal Sig_{raw}. FIG. 14 illustrates a preferred embodiment for calculating the average curve (baseline) BL. A segment of the raw signal Sig_{raw} is shown, which comprises the six heartbeats x₁, . . . , x₆. A sequence of segments Sig_{raw}(x_{n,n+1}), Sig_{raw}(x_{n+1,n+2}), . . . of the raw signal Sig_{raw} between each two consecutive heartbeat time periods H_Zr(xₙ) and H_Zr(xₙ₊₁), H_Zr(xₙ₊₁) and H_Zr(xₙ₊₂) is determined. The segment Sig_{raw}(x_{1,2}) therefore lies between the two heartbeat time periods H_Zr(x₁) and H_Zr(x₂) etc. Preferably, a specified time interval of Δt occurs between the period covered by the Sig_{raw}(x_{n,n+1}) segment and the two neighboring heartbeat time periods H_Zr(x₁) and H_Zr(xₙ₊₁).

For each segment Sig_{raw}(x_{1,2}), Sig_{raw}(x_{2,3}), an interpolation point (support point/base) Stp(1,2), Stp(2,3), . . . is determined. Stp(k,k+1) denotes the interpolation point for the segment Sig_{raw}(x_{k,k+1}) (k=1, 2, . . . ). A spline is drawn through this sequence of interpolation points Stp(1,2), Stp(2,3), . . . . Stp(k,k+1) denotes the interpolation point for the segment Sig_{raw}(x_{k,k+1}) (k=1, 2, . . . ). The segment of the spline between two neighboring interpolation points is a polynomial. Preferably, a Piecewise Cubic Hermite Interpolating Polynomial is used as the spline, with a third-order polynomial occurring between two neighboring interpolation points.

The following groups of quality indicators (quality assessments, quality indicators) can be distinguished. These groups lead to quality indicators Q[30], Q[31], Q[32], Q[33]. These quality indicators are calculated and applied in the initialization phase and/or in the use phase, which is described in more detail below.

Q[30]: How good is the sum signal Sig_{Sum} generated from the measured values of the sensors? Possible sources of error are:
The measured values of the sensors on or in or at the body of patient P are incorrect, for example due to poor or even lacking contact between a measuring electrode and the body of patient P, resulting in incorrect electrode resistance.
The measured values of the sensors are superimposed by disturbances, for example interference from a stationary power supply network, electro-galvanic interference (measuring electrode has been touched), other electromagnetic interference from electrical, or electronic devices in the vicinity.
The process of transmitting the measured values from the sensors to the signal processing unit 5 is subject to a fault, for example a cable break or a connection failure.
A technical fault occurs at the measured value processor 19, for example an out-of-range condition with an A-D converter, an empty battery, or an internal communication interruption.

The sum signal quality indicator Q[30] also includes how well the baseline BL was computationally removed.

Q[31]: With what reliability the heartbeat time period and/or the heartbeat time of a heartbeat have been detected in the sum signal Sig_{Sum}? Possible factors influencing the heartbeat time quality indicator Q[31] are
How regular is the heartbeat?
Are the courses or individual heartbeats sufficiently similar, see FIG. 1?
Have the signal segments for the individual heartbeats been found to be sufficiently valid?

Q[32]: what is the reliability that a reference attenuation signal segment Mod(1), . . . Mod(n) or an adapted attenuation signal segment Mod(1)(x), . . . , Mod(n)(x) is suitable for compensating for the computational contribution of the cardiogenic signal Sigₖₐᵣ to the intermediate signal Sig_{com}? This reliability can refer to a single heartbeat time period H_Zr(x), i.e. Can vary from heartbeat to heartbeat, or can refer to the reference heartbeat time period H_Zr_{ref} which is preferred. In one embodiment, and attenuation signal segment is derived from a power signal segment. Therefore, one embodiment for calculating the reliability Q[32] is as follows: Are a power signal segment Pow_{com}(i)(x) for a heartbeat x or an average power signal segment Pow_{com,av}(i) plausible, i.e. do they match expectations for power signal segment of a single heartbeat and/or for the expectations for an average power signal segment Pow_{com,av}(i)? With other words: How well does a power signal segment Pow_{com}(i)(x) or the average power signal segment Pow_{com,av}(i) describe the contribution of the cardiogenic signal Sigₖₐᵣ to the intermediate signal Sig_{com}(i) within the time period H_Zr(x) for a heartbeat x?

In one embodiment, a quality indicator Q[32] for a heartbeat x is calculated as a power quality indicator and serves as a weight factor in the process of calculating a weighted average over several power sample elements Pow_{com}(1), . . . , Pow_{com}(n) to derive an average power signal segment Pow_{com,av}(i). In one embodiment, the quality indicator Q[32] for an average power signal segment acts as an indicator of the reliability with which with which a reference attenuation signal segment Mod(i) for a frequency band (level) i describes the contribution of the cardiogenic signal Sigᵣₑₛ to the intermediate signal segment SigA_{com}(x), SigA_{com}(y) for a heartbeat period H_Zr(x), H_Zr(y) or for the reference heartbeat period H_Zr_{ref}.

Q[33]: does the actual cardiogenic signal segment SigAₖₐᵣ(x) fit to given expectations for a cardiogenic signal segment? The actual cardiogenic signal segment SigAₖₐᵣ(x) it's generated depending on a cardiogenic signal segment SigA_{kar,ref} or SigAₖₐᵣ(x). Therefore, one embodiment is as follows: Does the cardiogenic reference signal segment SigA_{kar,ref}, which is used for every heartbeat, or the cardiogenic signal segment SigAₖₐᵣ(x) adapted for a heartbeat x match given expectations for a cardiogenic signal segment? In particular: Does the adapted cardiogenic signal segment SigAₖₐᵣ(x) have a time course from Q to T as shown in FIG. 1?

FIG. 7 and FIG. 12 show three additional function blocks that calculate these quality indicators Q[30], Q[31], Q[32], Q[33]:
A function block 130 evaluates the quality with which the sum signal Sig_{Sum} was generated. Function block 130 provides the quality indicator Q[30], which comprises a value for each heartbeat x.
A function block 131 provides the quality indicator Q[31], which comprises one respective value for each heartbeat x.
A function block 132 provides the quality indicators Q[32] and Q[33]. In one embodiment, the respective quality indicator Q[32] comprises a single value for each heartbeat x; in another embodiment, it comprises a time course of values. The same applies to the quality indicator Q[33].

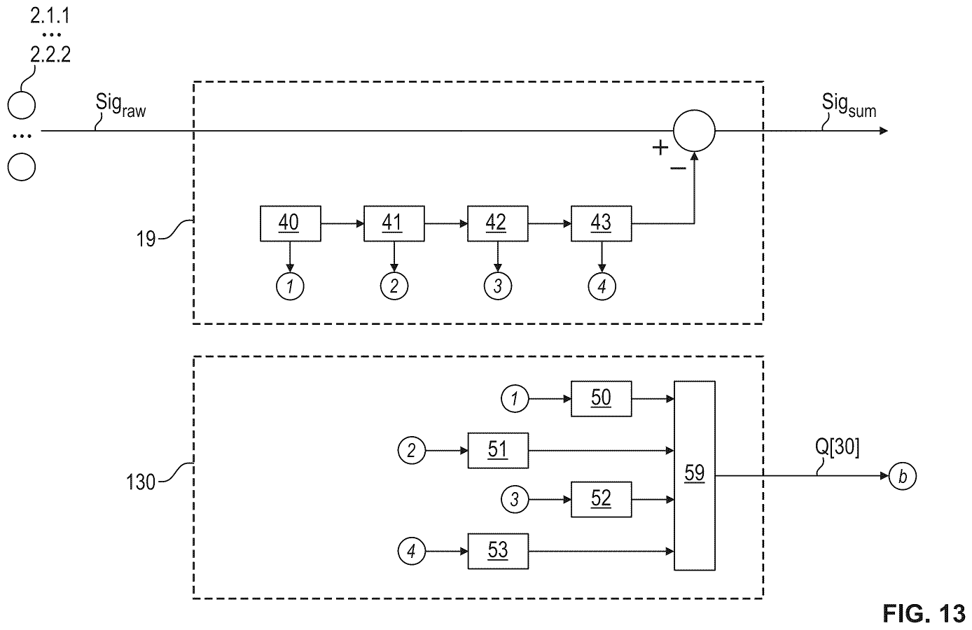

FIG. 13 shows an example of several functional units of the measured value processor 19 as well as the function block 130, which evaluates the quality Q[30] with which the measured value processor 19 generates (derives) the sum signal Sig_{Sum} from the raw signal Sig_{raw}.

It is possible that the heart activity of the patient P acts on (and is present in) at least two different sum signals, in particular on the respective sum signal from different sensors. In one embodiment, different heartbeat time points are detected. However, they all originate from the same heart and are therefore different estimates for the same event. In one embodiment, a heartbeat time point H_Zp(x), H_Zp(y) is selected from a signal. In one embodiment, the function block 131 evaluates how far the estimates for a heartbeat time point H_Zp(x), H_Zp(y) differ from each other and determines the quality indicator Q[31] depending on the differences.

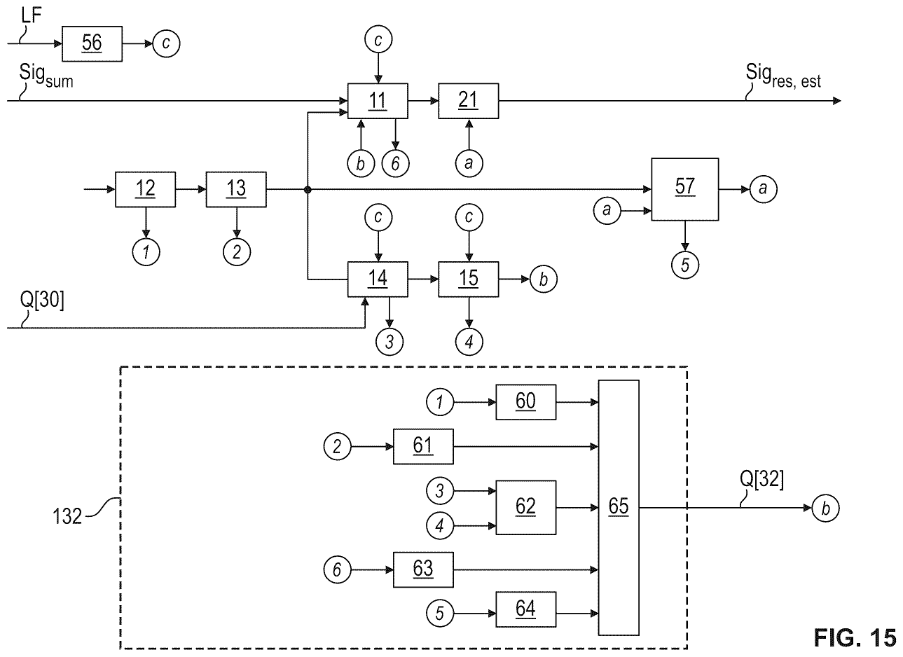

FIG. 15 shows the function block 132 in detail. As already explained, the function block 132 evaluates whether the determined cardiogenic reference signal segment SigA_{kar,ref} or the adapted cardiogenic signal segment SigAₖₐᵣ(x) for a heartbeat x matches specified expectations for a cardiogenic signal segment. The function block 132 provides a quality indicator Q[33]. In addition, the function block 132 determines the quality indicator Q[32].

The following functional units are shown in FIG. 15:
As already mentioned, the functional unit 12 identifies the respective heartbeat time period H_Zr(x) of each heartbeat x, preferably the QRS phase, in the sum signal Sig_{Sum}.
The functional unit 13 determines the respective characteristic heartbeat time point H_Zp(x) of each heartbeat x.
As already mentioned, the functional unit 14 superimposes the N sum signal segments SigA_{Sum}(x₁), . . . , SigA_{Sum}(x_{N}) for the last N heartbeats x₁, . . . , x_{N}.
The functional unit 15 generates a cardiogenic reference signal segment SigA_{kar,ref} from the superposition of N sum signal segments SigA_{Sum}(x₁), . . . , SigA_{Sum}(x_{N}).
An optional functional unit 56 determines a shape-changing factor for the cardiogenic reference signal segment SigA_{kar,ref} depending on a value of an anthropological parameter of the patient P during the heartbeat x and thereby generates an adapted cardiogenic signal segment SigAₖₐᵣ(x) for a heartbeat x. The anthropological parameter is, for example, the current lung filling level or the current position of the patient P. An exemplary mode of operation of this functional unit is described in DE 10 2019 006 866 A1 and US 2022/0 330 837 A1.
The functional unit 11 subtracts the cardiogenic reference signal segment SigA_{kar,ref} or an adapted cardiogenic signal segment SigAₖₐᵣ(x) from the sum signal Sig_{Sum}.
In order to remove remaining components of the cardiogenic signal Sigₖₐᵣ, the optional attenuation function block 21 performs an attenuation of the cardiogenic reference signal segment SigA_{kar,ref} or of the adapted cardiogenic signal segment SigAₖₐᵣ(x).
The functional unit 57 performs a residual power analysis and exchanges signals with the attenuation function block 21 for this purpose. In such a residual power analysis, the signal strengths of attenuation signal segments Mod(i) are examined and, optionally, an attenuation is performed. The attenuation was described by way of example with reference to FIG. 7 to FIG. 11.

The functional units 12, 13, 16 and 21 perform the respective calculation steps with a high sampling frequency of a few milliseconds, so that the respective result is already available during the respective heartbeat. The functional units 14, 15 and 57 perform the calculation steps with a lower sampling frequency, e.g. the low sampling frequency mentioned above, and process the N sum signal segments SigA_{Sum}(x₁), . . . , SigA_{Sum}(x_{N}) of N already completed heartbeats.

As already mentioned above, an average power signal segment Pow_{com,av}(i) is calculated, see FIG. 12. This average power signal segment Pow_{com,av}(i) describes the time course of an electrical power for level no. i, wherein the time course covers a single relative heartbeat time period T. The average power signal component Pow_{com,av}(i) is calculated as a weighted average over the M signal component segments SigA_{com}(i) of M heartbeats. A function block 101 positions the M signal component segments SigA_{com}(i) in the correct time relative to each other with respect to the reference heartbeat time period H_Zr_{ref}. The functional unit 24 generates the average power signal segment Pow_{com,av}(i) from the time-correctly positioned M signal component segments SigA_{com}(i) and from this the reference attenuation signal segment Mod(i), as described above.

The functional unit 24 of FIG. 7 and FIG. 12 uses at least one of the four quality indicators Q[30] to Q[34] for the following tasks:
in the initialization phase for the task of calculating the weight factor that are used to calculate the respective average power signal segment Pow_{com,av}(i) for each level no. i,
also in the initialization phase for the task of generating the reference attenuation signal segment Mod(i),
in the use phase for the optional task of updating the reference attenuation signal segment Mod(i), and
in the use phase for the task of calculating the adapted attenuation signal segment Mod(i)(x) for a heartbeat x by using the reference attenuation signal segment Mod(i)(x).

For each one of these tasks, the functional unit 24 determines an overall quality indicator Q and uses at least one quality indicator Q[30] to Q[34] for this calculation, preferably several quality indicators. One rule is that the lower a used and above-mentioned weight factor is, the worse the overall quality indicator Q is. In addition, the adapted attenuation signal segment Mod(i)(x) is smaller than or at most the same size as the reference attenuation signal segment Mod(i) and the smaller the worse the overall quality indicator Q is.

In the use phase, the signal processing unit 5 uses the previously determined reference attenuation signal segment Mod(i) for a heartbeat x and for a level no. i to generate an adapted attenuation signal segment Mod(i)(x). One effect of this attenuation is as follows: In the use phase, the attenuation is amplified if and as long as a poor overall quality indicator Q has been determined. A signal segment of the signal Sig_{com} with a poor quality Q is therefore attenuated more in comparison to other signal segments.

In particular, the following implementations are possible, as the adapted attenuation signal segment Mod(i)(x) is changed in the use phase depending on the overall quality indicator Q:
Each value of the adapted attenuation signal segment Mod(i)(x) is multiplied by a factor α<1, wherein this factor α<1 is derived from the quality indicator Q[30], Q[31], Q[32], Q[33] and is smaller the worse the overall quality indicator Q is.
Only those values of the adapted attenuation signal segment Mod(i)(x) are multiplied by the factor α<1 that are less than or equal to a specified upper bound β, where: 0<β<1.
Each value of the adapted attenuation signal segment Mod(i)(x) is reduced by a fixed value Δ>=0, wherein the worse the overall quality indicator Q is, the greater the fixed value Δ is. However, the value of the adapted attenuation signal segment Mod(i)(x) is reduced to a maximum of 0, i.e. no negative values occur.
Only those values of the adapted attenuation signal segment Mod(i)(x) are reduced by the fixed value Δ that are less than or equal to the upper bound β mentioned above.
Each value of the adapted attenuation signal segment Mod(i)(x) is multiplied by the factor α or reduced by the fixed value Δ, depending on which procedure leads to the smaller result. Again, values less than zero are avoided.

In addition to or instead of the implementations just mentioned, the adapted attenuation signal segment Mod(i)(x) is computationally smoothed, in particular by applying a moving average filtering.

The factor α just mentioned and/or the fixed value Δ just mentioned can be the same for each level i, i.e. for each frequency band. It is also possible that up to three individual quality indicators Q[30](i), Q[31](i), Q[32](i) are determined for each level i (i=1, . . . , n) in the use phase and a separate overall quality indicator Q=Q(i) is derived from this. Accordingly, a separate factor α(i) and/or a separate fixed value Δ(i) are derived for each level i and used as just described.

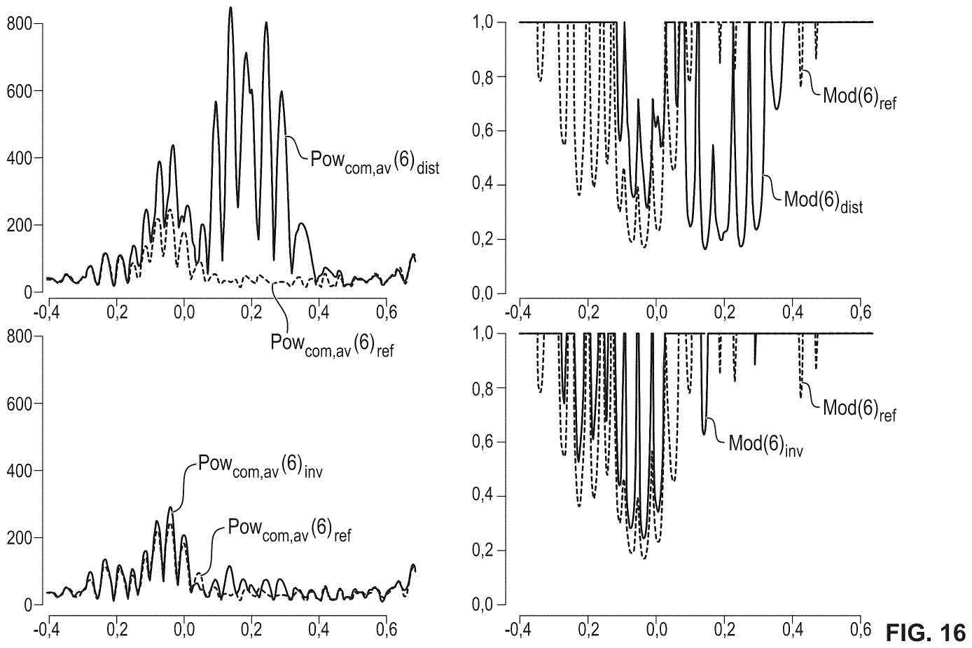

FIG. 16 illustrates an improvement achieved by the invention. The left-hand column examples of time courses of the averaged power signal segment Pow_{com,av}(6) for level 6, while the right-hand column shows time courses of the resulting reference attenuation signal segment Mod(6). The top row illustrates a result of a process which does not use the invention, the bottom row the result of the process according to the invention. Each diagram shows a time course in a situation free of interference and a time course in the presence of interference. In detail

| | |
|---|---|
| Pow_{com, av}(6)_{dist} | averaged power signal segment in the presence of |
| | interference, according to the state of the art, |
| Pow_{com, av}(6)ᵢₙᵥ | averaged power signal segment in the presence of |
| | interference, according to the invention, |
| Pow_{com, av}(6)_{ref} | averaged power signal segment without interference, |
| Mod(6)_{dist} | reference attenuation signal segment in the presence of |
| | interference, according to the state of the art, |
| Mod(6)ᵢₙᵥ | reference attenuation signal segment in the presence of |
| | interference is achieved according to the invention, |
| Mod(6)_{ref} | Reference attenuation signal segment without |
| | interference. |

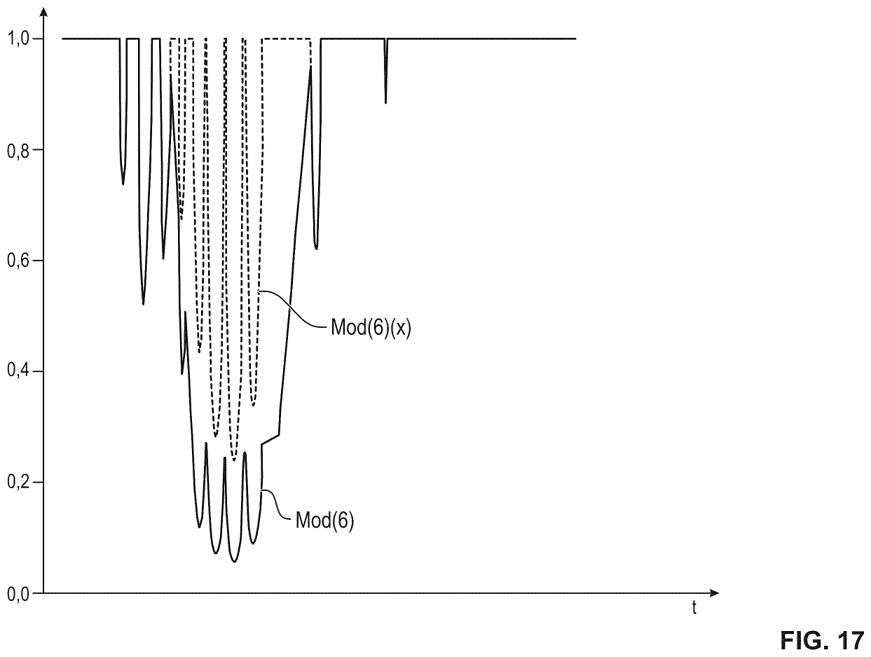

FIG. 17 illustrates an example of a reference attenuation signal segment Mod(6) and an adapted attenuation signal segment Mod(6)(x) for the heartbeat x. The time t is plotted on the x-axis and the signal value, which lies between 0 and 1, is plotted on the y-axis.

While specific embodiments of the invention have been shown and described in detail to illustrate the application of the principles of the invention, it will be understood that the invention may be embodied otherwise without departing from such principles.

### LIST OF REFERENCE SYMBOLS

| | |
|---|---|
| 1 | Ventilator, artificially ventilates and/or monitors the patient P, |
| | comprises the signal processing unit 5 |
| 2.1 | Intercostal (near the heart) pair of measuring electrodes on the |
| | patient's skin P, provides measured values for the electrical sum |
| | signal Sig_{Sum} |
| 2.1.1, 2.1.2, | Measuring electrodes of the intercostal pair 2.1 |
| 2.2 | Pair of measuring electrodes close to the diaphragm on the patient's |
| | skin P, provides further measured values for the sum signal Sig_{Sum} |
| 2.2.1, 2.2.2 | Measuring electrodes of the pair of electrodes close to the |
| | diaphragm 2.2 |
| 3 | Pneumatic sensor in front of the patient's mouth P, measures the |
| | volume flow Vol′ and the airway pressure P_{aw} |
| 4 | Optical sensor with an image acquisition device and an image |
| | processing unit, measures the geometry of the patient's body P, |
| | from which the current lung filling level Vol is calculated |
| 5 | Signal processing unit, carries out the steps of the process |
| | according to the invention, has read access and write access to the |
| | data memory 9 |
| 6 | Probe in the esophagus Sp, measures the pneumatic pressure Pₑₛ in |
| | the esophagus Sp |
| 7 | Cuff around one wrist of the patient P, holds the catheter 17, which |
| | invasively measures the time course of the blood pressure |
| 8.1 | Sensor in the form of a finger clip on a patient's finger P, measures |
| | the degree of saturation of the blood with oxygen non-invasively |
| 8.2 | Sensor in the form of a finger clip on another finger of patient P, |
| | non-invasively measures patient P's blood pressure |
| 9 | Data memory to which the signal processing unit 5 has at least |
| | temporary read access and write access and in which the |
| | cardiogenic reference signal segment SigA_{kar, ref} and the respiratory |
| | reference attenuation signal segments Mod(i) are stored |
| 10 | Functional unit of the compensation function block 20: generates |
| | the synthetic cardiogenic signal Sig_{kar, syn} |
| 11 | Functional unit of the compensation function block 20: |
| | compensates for the influence of the cardiogenic signal Sig_{kar, syn} on |
| | the sum signal Sig_{Sum} using the synthetic cardiogenic signal |
| | Sig_{kar, syn}, for example by subtracting Sig_{kar, syn} |
| 12 | Functional unit of the signal processing unit 5: recognizes the |
| | respective QRS time duration (QRS segment) of heartbeat in the |
| | sum signal Sig_{Sum} |
| 13 | Functional unit of the signal processing unit 5: detects the exact |
| | heartbeat time H_Zp(n) of each heartbeat |
| 14 | Functional unit of the compensation function block 20: |
| | superimposes N sum signal segments SigA_{Sum}(x₁), . . . , SigA_{Sum}(x_{N}) |
| | for the last N heartbeats |
| 15 | Functional unit of the compensation function block 20: generates a |
| | cardiogenic reference signal segment SigA_{kar, ref} |
| 16 | Functional unit of the compensation function block 20: positions |
| | the cardiogenic reference signal segments SigA_{kar, ref} or the adapted |
| | cardiogenic signal segments SigAₖₐᵣ(x) according to the time of the |
| | heartbeat H_Zp(x), combines the positioned Cardiogenic reference |
| | signal segments SigA_{kar, ref} to the synthetic cardiogenic signal |
| | Sig_{kar, syn} |
| 17 | Catheter, held by the cuff 7, invasively measures the time course of |
| | the patient's blood pressure |
| 19 | Measured value processor generates the sum signal Sig_{Sum} from the |
| | measured values of the measuring electrodes 2.1.1 to 2.2.2 |
| 20 | Compensation function block: generates the synthetic cardiogenic |
| | signal Sig_{kar, syn} and the compensation signal Sig_{com} |
| 21 | Attenuation function block: generates the estimated respiratory |
| | signal Sig_{res, est} from the compensation signal Sig_{com} by attenuation |
| 22 | Functional unit of the attenuation function block 21: decomposes |
| | (breaks down) the compensation signal Sig_{com} into n signal |
| | component segments SigA_{com}(1)(x), . . . , SigA_{com}(n)(x) for n levels |
| 23 | Functional unit of the attenuation function block 21: generates the |
| | estimated respiratory signal Sig_{res, est} from the compensation signal |
| | Sig_{com} by attenuation |
| 23(i) | Functional unit of the attenuation function block 21: generates the |
| | attenuated signal component segment SigA_{com, d}(i) (i = 1, . . . , n) from |
| | the signal component segment SigA_{com}(i) |
| 24 | Functional unit of the attenuation function block 21: generates the |
| | reference attenuation signal segment Mod(i) (i = 1, . . . , n) |
| 25 | Functional unit of the attenuation function block 21: composes |
| | (combines) the attenuated signal component segments SigA_{com, d}(1), |
| | . . . , SigA_{com, d}(n) by reverse transformation (back-transformation) to |
| | the newest segment SigA_{com, d} of the attenuated compensated signal |
| | SigA_{com, d}, this segment being used as the newest segment of the |
| | estimated respiratory signal Sig_{res, est} |
| 26 | Functional unit in the functional unit 23 / 23(i): applies the |
| | reference attenuation signal Mod(i) to the signal component |
| | SigA_{com}(i)(x) and generates the attenuated signal component |
| | SigA_{com, d}(i)(x) (i = 1, . . . , n) |
| 30 | Functional unit of the attenuation function block 21: generates the |
| | compensation signal segments from the compensation signal Sig_{com} |
| | using the characteristic heartbeat times |
| 32 | Optional functional unit: delays the sum signal Sig_{Sum} for the |
| | runtime required to determine the characteristic heartbeat time |
| | H_Zp(x) |
| 40 | Functional unit: detects the QRS segment in the raw signal Sig_{raw}, |
| 41 | Functional unit: detects the length of a currently evaluated segment |
| | in the raw signal Sig_{raw} |
| 42 | Functional unit: detects an interpolation point in the currently |
| | evaluated segment |
| 43 | Functional unit: constructs a spline for each heartbeat by |
| | interpolation |
| 50 | Functional unit: evaluates the regularity with which the functional |
| | unit 40 detects the QRS segments |
| 51 | Functional unit: detects evaluation segments that are particularly |
| | long or particularly short |
| 52 | Functional unit: determines the standard deviation of the random |
| | variable |
| 53 | Functional unit: evaluates the changes between the splines of two |
| | immediately consecutive heartbeats |
| 56 | Functional unit: determines a shape-changing factor for the |
| | cardiogenic reference signal segment SigA_{kar, ref} depending on the |
| | current lung filling level and thus generates a cardiogenic signal |
| | segment SigAₖₐᵣ(x) for a heartbeat x |
| 57 | Optional functional unit: analyzes the residual power |
| 59 | Functional unit: determines the quality indicator Q[30] from the |
| | individual quality indicators of the functional units 50 to 53 |
| 60 | Functional unit: evaluates the quality with which the functional unit |
| | 13 detects the exact heartbeat time H_Zp(x) of each heartbeat x |
| 61 | Functional unit: evaluates the quality with which a cardiogenic |
| | reference signal segment SigA_{kar, ref} or an adapted cardiogenic signal |
| | segment SigAₖₐᵣ(x) is generated for the heartbeat x |
| 62 | Functional unit: evaluates the quality with which the functional unit |
| | 16 subtracts the cardiogenic reference signal segment SigA_{kar, ref} or |
| | the adapted cardiogenic signal segment SigAₖₐᵣ(x) from the sum |
| | signal Sig_{Sum} |
| 63 | Functional unit: evaluates the quality with which the functional unit |
| | 57 has analyzed the residual power |
| 64 | Functional unit: determines the quality indicator Q[32] |
| 101 | Function block, positions the M signal component segments |
| | SigA_{com}(i) in the correct time relative to each other with respect to |
| | the reference heartbeat time period H_Zr_{ref} |
| 130 | Function block: evaluates the quality with which the sum signal |
| | Sig_{Sum} was generated from the raw signal Sig_{raw}, provides the |
| | quality indicator Q[30], comprises the functional units 50 to 53 and |
| | 59 |
| 131 | Function block: evaluates the reliability with which the |
| | characteristic heartbeat time point H_Zp(x) was detected by |
| | evaluating the sum signal Sig_{Sum}, provides the quality indicator |
| | Q[31], comprises the functional units |
| 132 | Function block: evaluates the quality with which the cardiogenic |
| | reference signal segment SigA_{kar, ref} or the determined cardiogenic |
| | signal segment SigA_{kar, syn}(x) was determined for a heartbeat x, |
| | provides the quality indicator Q[32], comprises the functional units |
| | 60 to 65 |
| Act | Optional update in the use phase of the reference attenuation signal |
| | segment Mod(i) |
| Atm(1), . . . | Time durations of breaths |
| Avg(i) | Average signal value for level no. i (i = 1, . . . , n), calculated from the |
| | averaged power signal segment Pow_{com, av}(i) (i = 1, . . . , n) |
| H_Zp(x), | Characteristic heartbeat time point of the heartbeat x or, detected by |
| H_Zp(y) | the functional unit 13 |
| H_Zr(x), | Heartbeat time period of the heartbeat x or y |
| H_Zr(y) | |
| H_Zr_{ref} | Reference heartbeat time period, covered by the cardiogenic |
| | reference signal segment SigA_{kar, ref} and by the reference attenuation |
| | signal segment Mod(i) |
| M | Number of heartbeats used to calculate the average power signal |
| | segment Pow_{com, av}(i) for level no. i in the initialization phase |
| Mod(i) | Reference attenuation signal segment for level no. i, covers the |
| | reference heartbeat time period H_Zr_{ref} |
| Mod(i)(x) | Adapted attenuation signal segment for level no. i and heartbeat x, |
| | covers the heartbeat time period H_Zr_{ref} |
| n | Number of levels (frequency bands) into which the compensation |
| | signal Sig_{com} is broken down |
| N | Number of heartbeats used in the initialization phase to generate the |
| | cardiogenic reference signal segment SigA_{kar, ref} |
| P | Patient is artificially ventilated with the aid of ventilator 1 |
| P_{aw} | Indicator for the airway pressure (pressure in airway) |
| Pₑₛ | Indicator for the esophageal pressure (pressure in esophagus) |
| Pₘᵤₛ | Pneumatic indicator of the patient's own breathing activity P |
| Pow_{com}(i)(x) | Power signal segment for level no. i and for a heartbeat x |
| Pow_{com, av}(i) | Average power signal segment for level no. i, calculated as a |
| | weighted average of M individual power signal segments |
| | Pow_{com}(i)(x), covers a reference heartbeat time period H_Zr_{ref} |
| φ(i) | Threshold value (threshold) for level no. i (i = 1, . . . , n) |
| Q[30] | Quality indicator for the quality with which the sum signal Sig_{Sum} |
| | was generated from the measured values of sensors 2.1.1 to 2.2.2, |
| | calculated by function block 30 |
| Q[31] | Quality indicator for the reliability with which the characteristic |
| | heartbeat time point H_Zp(x) was detected, calculated by function |
| | block 31 |
| Q[32] | Quality indicator for the plausibility of an average power signal |
| | segment Pow_{com, av}(i), i.e. how well it matches a heartbeat and / or |
| | the expectations of an average power signal segment |
| Q[33] | Quality indicator for the quality with which the cardiogenic |
| | reference signal segment SigA_{kar, ref} or the determined cardiogenic |
| | signal segment SigA_{kar, syn}(x) for a heartbeat x was determined, |
| | calculated by function block 32 |
| Sig_{com} | Compensation signal, is generated by the compensation function |
| | block 20 by compensating the contribution of the synthetic |
| | cardiogenic signal Sig_{kar, syn} to the sum signal Sig_{Sum}, serves as the |
| | intermediate signal |
| SigA_{com}(x) | Segment of the compensation signal Sig_{com} for the heartbeat x |
| SigA_{com}(i)(x) | Signal component segment for the level no. i (i = 1, . . . , n) of the |
| | segment SigA_{com}(x) for the heartbeat x, generated by the functional |
| | unit 22 by decomposing the compensation signal Sig_{com} |
| Sig_{com, d} | Attenuated compensation signal Sig_{com} |
| SigA_{com, d}(x) | Attenuated signal component for the heartbeat x |
| SigA_{com.d}(i)(x) | Attenuated signal component segment for the level no. i (i = 1, . . . , n) |
| | and the heartbeat x, generated by the functional unit 23(i) |
| Sigₖₐᵣ | Actual cardiogenic signal, causes the cardiac activity of the patient |
| | P, estimated by the synthetic cardiogenic signal Sig_{kar, syn} |
| Sig_{kar, syn} | Synthetic cardiogenic signal, is an estimate for the cardiogenic |
| | signal Sigₖₐᵣ, generated by the functional unit 10 from the signal |
| | segments SigA_{kar, syn}(x) |
| SigA_{kar, syn}(x) | Segment for the heartbeat x of the synthetic cardiogenic signal |
| | Sig_{kar, syn} |
| SigA_{kar, ref} | Cardiogenic reference signal segment, describes approximately the |
| | course of the cardiogenic signal Sigₖₐᵣ during a single heartbeat, |
| | refers to the reference heartbeat time period H_Zr_{ref} |
| Sig_{raw} | Raw signal from the measuring electrodes 2.1.1 to 2.2.2 |
| Sig_{raw}(x_{k, k+1}) | Segment of the raw signal Sig_{raw} between the two heartbeat time |
| | periods H_Zr(xₖ) and H_Zr(xₖ₊₁) |
| Sigᵣₑₛ | Respiratory signal to be determined, causes the patient's own |
| | breathing activity P |
| Sig_{res, est} | Estimate for the respiratory signal Sigᵣₑₛ to be determined according |
| | to the invention |
| Sig_{Sum} | Electrical sum signal, generated by the signal processing unit 5, |
| | comprises a superposition of the respiratory signal Sigᵣₑₛ with the |
| | cardiogenic signal Sigₖₐᵣ |
| SigA_{Sum}(x) | Segment of the sum signal Sig_{Sum} for the heartbeat time period |
| | H_Zr(x) of the heartbeat x |
| Stp(k, k + 1) | Interpolation point for the segment Sig_{raw}(x_{k, k+1}) |
| Sp | Esophagus of the patient P |
| τ | Time in the reference heartbeat time period H_Zr_{ref} |
| Vol′ | Volume flow |
| Tw | Diaphragm of the patient P |

What is claimed is:

1. A signal processing unit for determine a respiratory signal estimate, wherein a reference heartbeat time period and a use phase are specified, the signal processing unit being configured:
to generate a sum signal from received measured values of a sum signal sensor arrangement, which comprises one or more sum signal sensors, each sensor configured to measure a signal generated in and/or on the body of the patient, wherein the sum signal comprises a superposition of the respiratory signal to be estimated and a cardiogenic signal, which respiratory signal to be estimated correlates with the ventilation of the lungs of a patient and the ventilation of the lungs is caused by the patient's own breathing activity and/or by artificial ventilation of the patient and which cardiogenic signal correlates with the patient's cardiac activity;
by using the sum signal, to detect several heartbeats and for each detected heartbeat, to detect a characteristic heartbeat time period in which the detected heartbeats take place;
to determine an intermediate signal by at least partially compensating for an influence of the cardiac activity on the sum signal;
to determine at least one reference attenuation signal segment, in a first alternative, by calculation or, in a second alternative, by access to a data memory, wherein the reference attenuation signal segment correlates with an average time course of a contribution of the cardiogenic signal to the intermediate signal in the specified reference heartbeat time period;
for each heartbeat that is detected within the use phase, to generate a respective intermediate signal segment as a segment of the intermediate signal, wherein each generated intermediate signal segment lies in the heartbeat time period of the respective heartbeat;
for each detected heartbeat, to generate from each intermediate signal segment an attenuated intermediate signal segment lying in the heartbeat time period for the heartbeat, wherein an influence of the cardiogenic signal on the attenuated intermediate signal segment is less than or at most equal to an influence of the cardiogenic signal on the intermediate signal segment;
to form the estimation of the respiratory signal by combining the attenuated intermediate signal segments and using for the combination the detected characteristic heartbeat time periods;
the signal processing unit further being configured:
for each detected heartbeat, to apply the reference attenuation signal, in the first alternative, or an adapted reference attenuation signal segment, in the second alternative, to the intermediate signal segment to generate the respective attenuated intermediate signal segment;
the signal processing unit further being configured:
to determine at least one quality indicator selected from a set of quality indicators, the set comprising: a quality indicator for a reliability with which the sum signal sensor arrangement measures the respective measured values and/or a reliability with which the signal processing unit generates the sum signal from the respective measured values; for at least one heartbeat a respective quality indicator for a reliability with which a respective characteristic heartbeat time point of the heartbeat has been detected; a quality indicator for the reliability with which a reference attenuation signal segment compensates for the contribution of the cardiogenic signal to the intermediate signal in the reference heartbeat time period; and a quality indicator for the shape of the intermediate signal segment for a heartbeat; and
to determine, in the first alternative, the reference attenuation signal segment, using at least one quality indicator or
to determine, in the second alternative, for each heartbeat detected in the use phase, the respective adapted attenuation signal segment by using the determined reference attenuation signal segment and at least one quality indicator such that the determined adapted attenuation signal segment is smaller than or at most as large as the reference attenuation signal segment and the smaller the at least one quality indicator that is used is, the smaller the determined adapted attenuation signal segment is.

2. A signal processing unit according to claim 1, wherein the signal processing unit is configured to
generate a sample with a plurality of sample elements such that each sample element relates to a respective heartbeat and comprises the respective intermediate signal segment, and
generate a for each sample element respective power indicator sample element, the power indicator sample element comprising a time course of an indicator for the electrical power in the heartbeat time period of the heartbeat,
for the calculation of each reference attenuation signal segment, to generate an average power signal segment as an average over the power indicator sample elements, to determine the reference attenuation signal segment using the average power signal segment, and to cause the reference attenuation signal segment to be stored in the data memory.

3. A signal processing unit according to claim 2, wherein the signal processing unit is configured:
to determine, in the first alternative, the average over the power indicator sample elements as a weighted average using several weight factors,
to determine a weight factor for determining the average power signal segment using a respective power quality indicator for each power indicator sample element such that the smaller the power quality indicator is, the smaller the weight factor is.

4. A signal processing unit according to claim 3,
wherein the quality indicator, which is used to determine the weight factor, comprises an indicator of a reliability with which a reference attenuation signal segment compensates for the contribution of the cardiogenic signal to the intermediate signal in the reference heartbeat time period.

5. A signal processing unit according to claim 1,
wherein several frequency bands are specified,
wherein the signal processing unit is configured to determine, for each specified frequency band, a respective component of the reference attenuation signal segment for the frequency band or to determine the respective reference attenuation signal segment component by read access to the data memory,
wherein the signal processing unit is configured, for each specified frequency band and for each detected heartbeat that is within the use phase, to determine an adapted respective reference attenuation signal segment component for this heartbeat and for this frequency band by using the specified or determined reference attenuation signal segment component for this frequency band and at least one quality indicator,
wherein the adapted attenuation signal segment component correlates with the average time course of the contribution of the cardiogenic signal in the frequency band to the intermediate signal in the heartbeat time period, and
wherein the signal processing unit is further configured, for each specified frequency band and for each detected heartbeat that is within the use phase, to generate a respective intermediate signal segment component occurring in this frequency band and in the heartbeat time period of this heartbeat, and to generate a component of the attenuated intermediate signal segment for the heartbeat time period occurring in the frequency band by using the adapted respective reference attenuation signal segment component for this heartbeat and for this frequency band and by using the adapted attenuation signal segment component for this frequency band.

6. A signal processing unit according to claim 1, in combination with a sum signal sensor arrangement, which arrangement comprises one or more sum signal sensors, each sensor configured to measure a signal generated in or on the body of the patient, to provide a signal processing unit and sensor arrangement.

7. A process for determining an estimate for a respiratory signal, wherein the respiratory signal correlates with the ventilation of the lungs of a patient and the ventilation of the lungs is caused by the patient's own breathing activity and/or by artificial ventilation of the patient, the process comprising:
specifying a reference heartbeat time period and a use phase,
with a signal processing unit, receiving measured values from a sum signal sensor arrangement comprising at least one sum signal sensor, each sensor measures a signal generated in and/or on the body of the patient
with the signal processing unit, generating a sum signal using at least some of the received measured values, wherein the sum signal comprises a superposition of the respiratory signal to be estimated and a cardiogenic signal, which correlates with the patient's cardiac activity;
with the signal processing unit, using the sum signal to detect a plurality of heartbeats and, for each detected heartbeat, determining a characteristic heartbeat time period in which the heartbeat takes place;
with the signal processing unit, determining an intermediate signal by at least partially compensating for an influence of the cardiac activity on the sum signal;
with the signal processing unit, determining a reference attenuation signal segment, in a first alternative, by calculation or, in a second alternative, by a read access to a data memory, wherein the reference attenuation signal segment correlates with an average time course of a contribution of the cardiogenic signal to the determined intermediate signal in the specified reference heartbeat time period; the process further comprising, for each detected heartbeat that is within the use phase:
with the signal processing unit, generating a respective intermediate signal segment as a segment of the intermediate signal, which intermediate signal segment lies in the heartbeat time period of the heartbeat;
with the signal processing unit, generating from the intermediate signal segment an attenuated intermediate signal segment for the heartbeat time period, wherein an influence of the cardiogenic signal on the attenuated intermediate signal segment is less than or at most equal to the influence of the cardiogenic signal on the intermediate signal segment;
the process further comprising, with the signal processing unit, determining the estimate for the respiratory signal by combining the attenuated intermediate signal segments and using the detected characteristic heartbeat time periods,
the process further comprising:
for each detected heartbeat that is within the use phase, with the signal processing unit, applying the reference attenuation signal segment, in the first alternative, or applying an adapted attenuation signal segment, in the second alternative, to the respective intermediate signal segment to generate the attenuated intermediate signal segment for the heartbeat;
the process further comprising:
with the signal processing unit, determining at least one quality indicator selected from a set of quality indicators, the set comprising: a quality indicator for a reliability with which the sum signal sensor arrangement measures the respective measured values and/or a reliability with which the signal processing unit generates the sum signal from the respective measured values; for at least one heartbeat, a quality indicator for a respective reliability with which a respective characteristic heartbeat time point of a heartbeat has been detected for the heartbeat; a quality indicator for a reliability with which a reference attenuation signal segment compensates for a contribution of the cardiogenic signal to the intermediate signal in the reference heartbeat time period, and a quality indicator for a shape of the intermediate signal segment for a heartbeat; and
the process further comprising:
with the signal processing unit, in the first alternative, determining the reference attenuation signal segment, or in the second alternative, for each heartbeat detected in the use phase, using at least one quality indicator, determining the adapted attenuation signal segment for the heartbeat by using the determined reference attenuation signal segment and at least one quality indicator such that the determined adapted attenuation signal segment is smaller than or at most as large as the reference attenuation signal segment and such that the smaller the at least one quality indicator that is used is, the smaller the determined adapted attenuation signal segment is.

8. A process according to claim 7, further comprising:
with the signal processing unit, generating a sample with several sample elements such that each sample element relates to one heartbeat and comprises the intermediate signal segment as the segment of the intermediate signal, where the intermediate signal segment lies in the heartbeat time period of the heartbeat; and
generating a respective power indicator sample element for each sample element, the power indicator sample element comprising a time course of an indicator for the electrical power in the heartbeat time period of the heartbeat, and
wherein the signal processing unit determining the reference attenuation signal segment comprises the steps of: with the signal processing unit, generating an average power signal segment as an average over the power indicator sample elements, determining the reference attenuation signal segment using the average power signal segment, and causing the reference attenuation signal segment to be stored in the data memory.

9. A process according to claim 8,
wherein, in the first alternative, the average over the power indicator sample elements is determined as a weighted average using several weight factors,
wherein a weight factor for determining the average power signal segment is determined using the at least one quality indicator such that the smaller the quality indicator, the smaller the weight factor.

10. A process according to claim 9,
wherein the quality indicator used to determine the weight factor comprises an indicator of the reliability with which a reference attenuation signal segment compensates for the contribution of the cardiogenic signal to the intermediate signal segment in the reference heartbeat time period, and the at least one quality indicator is a quality indicator for a shape of the average power signal segment.

11. A process according to claim 7,
wherein several frequency bands are specified,
wherein the process further comprises the steps of:
with the signal processing unit, for each specified frequency band, determining a respective component of the reference attenuation signal segment or determining the respective component of the reference attenuation signal segment by a read access to the data memory, wherein the component refers to the frequency band;
with the signal processing unit, for each specified frequency band and for each detected heartbeat that falls within the use phase, generating a respective component of the intermediate signal segment for the heartbeat time period of the respective detected heartbeat occurring in the frequency band, and from the generated component, generating a component of the attenuated intermediate signal segment for the heartbeat time period of the respective detected heartbeat occurring in the respective frequency band;
with the signal processing unit, composing the attenuated intermediate signal segment for the heartbeat time period from the components for the frequency bands for each detected heartbeat which falls into the use phase,
with the signal processing unit, for each specified frequency band, for the generation of the component of the attenuated intermediate signal segments occurring in the frequency band, applying in the first alternative, the component of the reference attenuation signal segment for the frequency band, and in the second alternative, applying a component of the adapted attenuation signal segment for the frequency band, to the component of the intermediate signal component; and
with the signal processing unit, for each specified frequency band, in the first alternative, determining the respective component for the respective frequency band of the reference attenuation signal segment using at least one quality indicator, and in the second alternative, for each heartbeat detected in the use phase, determining the component of the attenuated intermediate signal segment occurring in the respective frequency band using the determined component of the respective frequency band of the reference attenuation signal segment and the at least one quality indicator, wherein said component of the attenuated intermediate signal segment is less than or at most equal to the reference attenuation signal segment for the frequency band and is smaller, the smaller a quality indicator used is.

12. A signal processing unit comprising:
a measured value processor, which is configured to generates a sum signal from measured values from a sum signal sensor arrangement, which arrangement comprises one or more sum signal sensors configured to measure a signal generated in and/or on the body of the patient, wherein the sum signal comprises a superposition of a respiratory signal and a cardiogenic signal, which respiratory signal correlates with the ventilation of the lungs of a patient and the ventilation of the lungs is caused by the patient's own breathing activity and/or by artificial ventilation of the patient and which cardiogenic signal correlates with the patient's cardiac activity;
a heartbeat functional unit, which is configured to detect a plurality of heartbeats by using the sum signal and, for each detected heartbeat, to determine a characteristic heartbeat time period, in which period the respective heartbeat takes place;
a compensation function block, which is configured to receive the sum signal and the characteristic heartbeat time periods and, based on the sum signal and the characteristic heartbeat time periods, to calculate an intermediate signal that at least partially compensates for an influence of the cardiac activity on the sum signal; and
an attenuation function block, which is configured:
to receive the intermediate signal and the characteristic heartbeat time periods;
to generate intermediate signal segments as segments of the intermediate signal, wherein each intermediate signal segment lies in a respective one of the heartbeat time periods;
to generate from the intermediate signal segments attenuated intermediate signal segments for the heartbeat time periods by applying a reference attenuation signal segment to the intermediate signal segments or by applying an adapted attenuation signal segment to the intermediate signal segment, wherein an influence of the cardiogenic signal on the attenuated intermediate signal segments is less than or equal to an influence of the cardiogenic signal on the intermediate signal segments; and
to combine the attenuated intermediate signal segments using the detected characteristic heartbeat time periods to form an estimation of the respiratory signal,
wherein the signal processing unit is configured to determine one or more quality indicators from quality indicators comprising a sum signal quality indicator; a heartbeat time quality indicator; a reference attenuation signal segment quality indicator; and an intermediate signal segment shape quality indicator,
wherein the reference attenuation signal segment is determined from a connected read access memory or is determined based on the determined one or more quality indicators,
wherein the attenuated intermediate signal segment is determined based on the determined reference attenuation signal segment and the determined one or more quality indicators or the adapted attenuation signal segment is determined based on the determined reference attenuation signal segment and the at least one quality indicator such that the determined adapted attenuation signal segment is smaller than or at most as large as the reference attenuation signal segment and the smaller the at least one quality indicator that is used is, the smaller the determined adapted attenuation signal segment is.

13. A signal processing unit according to claim 12,
wherein the signal processing unit is configured to generate a sample with several sample elements that each relate to a respective heartbeat and comprise the respective intermediate signal segment as the segment of the intermediate signal lying in the respective heartbeat time period of a heartbeat, and
wherein the attenuation function block is configured:
to generate for each sample element a respective power indicator sample element, the power indicator sample element comprising a time course of an indicator for electrical power in the heartbeat time period of the heartbeat;
to generate an average power signal segment as an average over the power indicator sample elements;
to determine each reference attenuation signal segment based on the average power signal segment; and
to store each determined reference attenuation signal segment in the data memory.

14. A signal processing unit according to claim 13, wherein the attenuation function block is configured:
to calculate the average over the power indicator sample elements as a weighted average; and
to calculate a weight factor for calculating the average power signal segment, wherein a power quality indicator for each power indicator sample element is formed such that a smaller the power quality indicator, the smaller the weight factor.

15. A signal processing unit according to claim 14,
wherein the power quality indicator used to calculate the weight factor, is the reference attenuation signal segment quality indicator and comprises a shape of the average power signal segment or the power indicator sample element.

16. A signal processing unit according to claim 12,
wherein several frequency bands are specified and
wherein the attenuation function block is configured:
to determine, for each specified frequency band, a reference attenuation signal segment by calculation or to determine the reference attenuation signal segment for each specified frequency band by the read access to the data memory;
to process the intermediate signal for each specified frequency band and for each detected heartbeat using the determined reference attenuation signal segment for each specified frequency band or the reference attenuation signal segment for each specified frequency band and the determined one or more quality indicators to calculate at least one adapted attenuation signal segment for each specified frequency, wherein the adapted attenuation signal segment correlates with an average time course of the contribution of the cardiogenic signal in the frequency band to the intermediate signal in the heartbeat time period; and
to generate a respective component of the intermediate signal segment occurring in this frequency band for the heartbeat time period of the detected heartbeat using the respective generated component occurring in this frequency band and the adapted attenuation signal segment for this frequency band.

17. A signal processing unit according to claim 12 in combination with at least one sum signal sensor to form a signal processing unit and sum signal sensor system.

18. A signal processing unit according to claim 17 in combination with a visualization display to form a signal processing unit, sum signal sensor system and display system.

19. A signal processing unit according to claim 12,
wherein a sum signal quality indicator function block provides the sum signal quality indicator value for each heartbeat from one or more functional units of the measured value processor and/or a determination or from an evaluation by the sum signal quality indicator function block of an output of one or more functional units of the measured value processor;
wherein a heartbeat time quality indicator function block provides the heartbeat time quality indicator based on heartbeat functional unit determination of a time duration of the heartbeat in the sum signal and a heartbeat time point functional unit that detects a time point of the heartbeat; and
wherein a functional block of the attenuation function block provides the reference attenuation signal segment quality indicator and the intermediate signal segment shape quality indicator.
